SEQUENCE LISTING (2 sequences)

SEQ ID NO: 1 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 2 - protein, 655 aa, synthetic construct
MAEAPQVVEIDPDFEPLPRPRSCAWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAAAMDNNSKFAKSRSRAAKKKASLQSGQEGAGDSPGSQFSKWPASPGSHSNDDFDNWSTFRPRTSANASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

(19) United States
(12) Patent Application Publication
(10) Pub. No.: US 2025/0249100 A1
(43) Pub. Date: Aug. 7, 2025

(54) COMPOSITIONS AND METHODS FOR PREVENTING T CELL EXHAUSTION

(71) Applicant: The Board of Trustees of the Leland Stanford Junior University, Stanford, CA (US)

(72) Inventors: Crystal Mackall, Stanford, CA (US); Evan Weber, Stanford, CA (US); Alexander E. Doan, Stanford, CA (US)

(21) Appl. No.: 18/855,242

(22) PCT Filed: Apr. 25, 2023
(86) PCT No.: PCT/US2023/066194

§ 371 Date: Oct. 8, 2024

Related U.S. Application Data
(60) Provisional application No. 63/334,462, filed on Apr. 25, 2022.

(51) Int. Cl. A61K 40/31; A61K 40/11; A61K 40/42; C07K 14/47; C07K 14/725; C07K 16/28; C07K 16/32; C12N 15/86

(52) U.S. Cl. CPC A61K 40/31; A61K 40/11; A61K 40/4205; A61K 40/4211; C07K 14/4702; C07K 14/7051; C07K 16/2803; C07K 16/32; C12N 15/86; C12N 2740/10043

(57) ABSTRACT

Provided herein are compositions comprising T cells modified to overexpress FOXO1 and methods of use thereof. Methods are provided to treat a disease or disorder in a subject comprising administration of the modified T cells. Also provided are methods for preventing exhaustion of engineered T cells comprising introducing a nucleic acid that overexpresses FOXO1 into the T cells.

### CROSS-REFERENCE TO RELATED APPLICATIONS

This application claims the benefit of U.S. Provisional Application No. 63/334,462, filed Apr. 25, 2022, the content of which is herein incorporated by reference in its entirety.

### FIELD

The present disclosure relates to compositions comprising T cells modified to overexpress FOXO1 and methods ofuse thereof.

### SEQUENCE LISTING STATEMENT

The contents of the electronic sequence listing titled “STDU2-39531-601_SQL.xml” (Size: 4,128 bytes; and Date of Creation: Apr. 24, 2023) is herein incorporated by reference in its entirety.

### BACKGROUND

The potency of chimeric antigen receptor (CAR) T cells is limited by T cell exhaustion, whereby chronic CAR signaling induces progressive loss of function. Exhaustion is often targeted with checkpoint inhibitors. However, a majority of patients fail to respond to these agents and no efficacy has been shown in combination with CAR T cells in clinical trials. Memory T cells, which are long-lived and highly proliferative progenitor cells that give rise to effector T cells upon re-encountering antigen, are vital for the anti-tumor immune response and are associated with responses to checkpoint blockade and adoptive T cell therapies. CAR T cells infused into patients lack high numbers of memory T cells. Poor CAR-T persistence and relapse in patients is associated with this deficit in memory CAR-T cell formation.

### SUMMARY

Provided herein are compositions comprising isolated T cells that comprise an exogenous nucleic acid encoding a forkhead box protein O1 (FOXO1). In some embodiments, the FOXO1 is wild-type FOXO1. In some embodiments, the FOXO1 is fully or partially nuclear-localized FOXO1. In some embodiments, FOXO1 is constitutively expressed from the exogenous nucleic acid. In some embodiments, FOXO1 is fused to a motif which modulates expression levels or enhances intracellular degradation (e.g., degron, destabilizing domain, or the like).

In some embodiments, the isolated T cells maintain functionality under conditions in which unmodified T cells display exhaustion.

In some embodiments, the isolated T cells further comprise a nucleic acid encoding a recombinant receptor. In some embodiments, the recombinant receptor is a T cell receptor (TCR) or a chimeric antigen receptor (CAR). In some embodiments, the recombinant receptor is specific for a tumor antigen.

In some embodiments, the FOXO1 and the engineered receptor are encoded by separate nucleic acids. In some embodiments, the FOXO1 and the engineered receptor are encoded by a single nucleic acid. In some embodiments, the FOXO1 and the engineered receptor are expressed under different promoters.

In some embodiments, the isolated T cells are from a biological sample from a subject. In some embodiments, the T cells are isolated from a tumor sample. In some embodiments, the T cells are expanded ex vivo.

In some embodiments, the composition further comprises at least one therapeutic agent.

Also provided herein are method of treating a disease or disorder in a subject comprising administering to the subject having the disease or disorder an effective amount of the disclosed composition. In some embodiments, the T cells are autologous to the subject. In some embodiments, the FOXO1 is overexpressed in the T cells prior to exposure to the antigen. In some embodiments, the method further comprises administering to the patient one or more chemotherapeutic agents.

In some embodiments, the disease or disorder comprises an infectious disease or cancer. In some embodiments, the cancer is a solid tumor. In some embodiments, the recombinant receptor is specific for the cancer.

In some embodiments, the administering reduces the number of cancerous cells in the patient. In some embodiments, the administering reduces and/or eliminates the tumor burden in the patient. In some embodiments, the administering shows enhanced cancer treatment compared to administration of unmodified T cells. In some embodiments, the administering results in lower expression of inhibitory receptors in the subject in comparison to administration of unmodified T cells.

Further provided herein are methods for preventing exhaustion of engineered T cells comprising introducing a nucleic acid that overexpresses FOXO1 into the engineered T cells. In some embodiments, the nucleic acid facilitates constitutive expression of FOXO1 from the nucleic acid.

In some embodiments, the FOXO1 is wild-type FOXO1. In some embodiments, the FOXO1 is fully or partially nuclear-localized FOXO1. In some embodiments, FOXO1 is fused to a motif which modulates expression levels or enhances intracellular degradation (e.g., degron, destabilizing domain, or the like).

In some embodiments, the engineered T cells comprise a nucleic acid encoding a recombinant receptor. In some embodiments, the recombinant receptor is a T cell receptor (TCR) or a chimeric antigen receptor (CAR). In some embodiments, the recombinant receptor is specific for a tumor antigen. In some embodiments, the FOXO1 is overexpressed prior to exposure to the antigen.

In some embodiments, the FOXO1 and the engineered receptor are encoded by separate nucleic acids. In some embodiments, the FOXO1 and the engineered receptor are encoded by a single nucleic acid. In some embodiments, the FOXO1 and the engineered receptor are expressed under different promoters.

In some embodiments, the methods further comprise administering the T cells to a subject in need thereof. In some embodiments, the subject has cancer.

Other aspects and embodiments of the disclosure will be apparent in light of the following detailed description.

### BRIEF DESCRIPTION OF THE DRAWINGS

FIGS. 1A-1C show use of destabilizing domain (DD)-modified CARs to interrogate exhaustion reversal. FIG. 1A is a schematic of the DD-CAR tunable platform. FIG. 1B is a dose-titration curve of GD2-28z-DD CAR. FIG. 1C is a schematic of HA-28z-DD in vitro model of CAR T cell exhaustion. CAR surface density is normalized across groups prior to functional assays on D15 (Weber et al., Science, 2021).

FIGS. 2A-2C show the expansion, viability (FIG. 2A), and phenotypic analyses of CAR+ cell surface and intracellular proteins carried out via flow cytometry (FIGS. 2B and 2C) for CD19.28z CAR-T cells expanded either in the presence or absence of the selective FOXO1 inhibitor AS1842856 (FOXO1i) for 16 days. FIG. 2A is a graph showing that FOXOi dose-dependently inhibits CAR-T cell expansion and viability during ex vivo expansion. FIG. 2B shows that FOXO1i dose-dependently reduced expression levels of FOXO1 target genes and increased expression of T cell effector/exhaustion markers. FIG. 2C shows FOXO1i dose-dependently reduced the frequency of memory-like (CD45RA+/CD62L+) CAR-T cell populations and increased the frequency of effector-like populations (CD45RA−/CD62L−). 1 representative donor from n=2 donors.

FIG. 3A is a schematic of an exemplary co-transduction approach as disclosed herein. Briefly, human T cells were first transduced with 1 retrovirus encoding a CAR. 24-hours later, CAR-T cells were then transduced with a separate retrovirus encoding either truncated NGFR alone (negative control) or bicistronic vectors that contain a transcription factor plus truncated NGFR. Other iterations of this approach include the inverse order (first TF, then CAR), as well as transductions wherein each virus is mixed at a 1:1 ratio. FIG. 3B shows intracellular flow cytometry of CD19.28z CAR-T cells co-transduced to express ectopic TFs. 1 representative donor from n=5 independent donors.

FIG. 4A shows phenotyping by flow cytometry on day 15 post-activation of CD19.BBz CAR-T cells +/−TFs. Control NGFR cells and those ectopically expressing TFs exhibit similar expression levels of exhaustion and differentiation markers. Similar data was obtained for CD19.28z CAR-T cells. Data is representative of n=5 independent donors. Day 15 CD19.28z CAR-T cells were unstimulated (Unstim) or crosslinked with an anti-FMC63 idiotype antibody plus a crosslinking goat anti-mouse secondary antibody for 5 hours (Stim) in the presence of monensin and an anti-CD107a antibody, which served as a surrogate readout for degranulation. Cells were then processed for flow cytometry and stained with an additional antibody against CD69, an early activation marker (FIG. 4B). Data demonstrates equivalent CD69 and CD107a expression in stimulated NGFR+ or NGFR− CAR-T cells. Similar data was obtained for CD19.BBz CAR-T cells. n=1 donor.

FIGS. 5A-5D are graphs of co-transduced CD19.28z (FIGS. 5A and 5B) or CD19.BBz (FIGS. 5C and 5D) CAR-T cells that underwent Miltenyi magnetic purification to isolate NGFR+ cells (using anti-NGFR-biotin antibodies and streptavidin magnetic beads) on day 11 post-activation. On Day 15, CAR+ NGFR+ cells were then co-cultured with Nalm6 cells engineered to express GFP and luciferase (Nalm6) at a 1:1 (FIGS. 5A and 5C) or 1:4 (FIGS. SB and 5D) E:T ratio. Cytokine secretion was determined via ELISA (FIGS. 5A and 5C) and cytotoxicity was determined via Incucyte assays ((FIGS. 5B and 5D). There were no detectable differences in cytokine secretion and killing at these E:T ratio across conditions. 1 representative donor from n=2 donors.

FIGS. 6A-6B show intracellular flow cytometry on CAR+/NGFR+/CD4+CD19.BBz CAR-T cells demonstrating that ectopic expression of FOXO1-WT and nuclear-restricted FOXO1-3A increases expression of endogenous LEF1 and TCF1. Similar data was obtained for the CD4+ subset as well as CD19.28z CAR-T cells. 1 representative donor from n=2 donors.

FIGS. 7A-7C show intracellular flow cytometry on CAR+/NGFR+/CD8+CD19.BBz CAR-T cells that were unstimulated (Unstim) or had been stimulated using the method described in FIG. 5 description (Stim). Stimulated FOXO1-WT cells demonstrated enhanced expression of endogenous LEF1, TCF1, T-bet, and Blimp-1 compared to NGFR control cells. Similar data was obtained for the CD4+ subset as well as for CD19.28z CAR-T cells. n=1 donor.

FIG. 8A show flow cytometry of phenotype of co-transduced CD19.BBz CAR-T cells challenged with Nalm6 at a 1:1 ratio either 0, 1, or 3 times and analyzed 7 days post-stimulation. CAR+/NGFR+/CD8+ FOXO1-WT and FOXO1-3A CAR-T cells maintained FOXO1 target gene expression and a memory-like cell subset (CD62L+/IL-7R+) after multiple rounds of tumor challenge, which was in contrast to CAR-T cells expressing NGFR alone or TCF1, which rapidly lost this memory-like subset. Similar data was obtained for the CD4+ subset and CD19.28z CAR-T cells (not shown). Representative donor from n=3 independent donors. FIGS. 8B and 8C are graphs of incucyte data from co-transduced CD19.28z or CD19.BBz CAR-T cells purified on NGFR+ cells and subsequently co-cultured with Nalm6 at a 1:16 E:T ratio showing that FOXO1-WT CAR-T cells maintain tumor killing to a greater extent than FOXO1-3A or NGFR control cells. FIG. 8D is a graph of NGFR+ CD19.28z CAR-T cells co-cultured with Nalm6 at a 1:4 E:T ratio and counted 5 days post-tumor challenge. Data shows enhanced expansion in FOXO1-WT cells. n=1 donor.

FIGS. 9A and 9B are flow cytometry phenotyping of Day 14 CAR+/NGFR+/CD8+HA.28z CAR-T cells showing decreased exhaustion marker expression and increased memory marker expression in FOXO1-WT and FOXO1-3A CAR-T cells compared to NGFR control cells. Similar results were obtained for the CD4+ subset. FIG. 9C is a graph of NGFR+ purified HA.28z CAR-T cells co-cultured with 143B osteosarcoma cells engineered to express GFP and luciferase (143B) at a 1:8 E:T. Incucyte data shows enhanced tumor killing in FOXO1-WT cells compared to FOXO1-3A and NGFR cells. FIGS. 9D and 9E are ELISA data from 1:1 E:T co-cultures with 143B (FIG. 9D) or Nalm6 (FIG. 9E) engineered to express GD2 (N6 GD2) shows enhanced IL-2 secretion from NGFR+ purified HA.28z FOXO1-WT cells compared to both FOXO1-3A and NGFR cells, and enhanced IFNy secretion compared to NGFR cells. Representative donor from n=2 donors.

FIG. 10A is flow cytometry results of human T cells transduced to express DD-FOXO1-WT co-cultured in various concentrations from trimethoprim (TMP) for 48 hours, which dose-dependently increased intracellular FOXO1-WT expression. n=1 donor. FIG. 10B is a graph of NGFR+ purified HA.28z CAR-T cells cultured in the presence of TMP starting on day 4-15 or day 14-15 and subsequently co-cultured with N6-GD2 at a 1:1 E:T ratio on day 15 in the presence of TMP. ELISA data on the co-culture supernatants demonstrates increased IL-2 secretion in CAR-T cells that were cultured with TMP from day 4-15, but not those cultured for TMP from day 14-15. Representative donor from n=2 donors.

FIG. 11A is a graph of the results of a Seahorse assay on co-transduced CD19.28z or HA.28z CAR-T cells purified on NGFR+ cells. Data demonstrates reduced basal ECAR and enhanced spare respiratory capacity (SRC) in FOXO1-WT and FOXO1-3A cells compared to NGFR control cells. n=1 donor. 1-way ANOVA and Dunnett's multiple comparisons test was used to determine statistical significance. ** p<0.01; **** p<0.0001. FIGS. 11B and 11C are flow cytometry data from co-transduced CD19.28z CAR-T cells stained with mitotracker green. Cells were gated on CAR+/NGFR+/CD8+. Data shows enhanced mitochondrial mass at baseline and after 24 hours of stimulation with Nalm6 (no GFRP or luciferase) at a 1:1 ratio in FOXO1-WT and FOXO1-3A cells compared to NGFR. Similar data was observed in the CD4+ subset. Representative donor from n=2 donors.

FIG. 12A is a graph of enhanced tumor control and survival in mice treated with FOXO1-WT and FOXO1-3A CAR-T cells compared to those treated with untransduced (mock), NGFR, or TCF1-expressing T cells. Nalm6 cells (1e6) were engrafted into NSG mice and a subtherapeutic dose of 0.1e6 CD19.28z+/NGFR+ T cells was infused 7 days post-engraftment. Data shows 5-11 mice per group from n=2 donors (TCF1 condition was only derived from 1 donor). Statistics were determined using log-rank Mantel-Cox test. FIG. 12B is a graph showing enhanced tumors in mice treated with FOXO1-WT and FOXO1-3A CAR-T cells compared to those treated with NGFR or TCF1-expressing T cells. Nalm6 (1e6) cells were engrafted into NSG mice and 1e6 CD19.BBz+/NGFR+ T cells were infused 7 days post-engraftment. Data shows 3-5 mice per group from n=1 donor. In FIG. 12C, the same experimental setup as in FIG. 12B was used, except cells were not purified on NGFR+ subset and only single transduced CD19.BBz or co-transduced CD19.BBz+FOXO1-WT cells were compared. FOXO1-WT cells again demonstrated enhanced tumor control at day 39-45 post-engraftment. Data represents 9-10 mice per group from n=2 donors. In FIG. 12D, HA.28z CAR-T cells or those co-transduced with FOXO1-WT were cultured ex vivo for 15 days. 1e6 CAR+ T cells (without NGFR+ purification) were subsequently infused into NSG mice 7 days post-engraftment of Nalm6-GD2. FOXO1-WT cells demonstrated tumor control on day 52 post-engraftment. Data represents 5 mice per group from n=1 donor. 1-way ANOVA and Dunnett's multiple comparisons test was used to determine statistical significance. * p<0.05; ** p<0.01

FIG. 13A is a schematic of an exemplary in vivo model. Briefly, 1e6 Nalm6 cells were engrafted into NSG mice and a therapeutic dose of CD19.28z+/NGFR+ T cells was infused 7 days later. Mice were rechallenged with 10e6 Nalm6 cells 21 days post-CAR-T infusion. Peripheral blood was harvested 7 days post-CAR-T infusion and every 7 days thereafter. FIG. 13B is a graph of Nalm6 bioluminescence data showing equivalent and near-complete tumor control in mice from each treatment group. FIGS. 13C and 13D are flow cytometry data showing similar levels of CAR and NGFR were observed on circulating human CD45+ T cells from day 14 post-CAR-T infusion. Representative mice from each group are displayed.

FIGS. 14A-14C show data derived from the experiment outlined in FIG. 13A. FIGS. 14A and 14B are graphs of total circulating CAR-T cell levels determined using flow cytometry and quantibright beads. FOXO1-WT cells demonstrated the most robust expansion in response to both the primary tumor challenge and the rechallenge. 3-7 mice/group from n=1 donor. IN FIG. 14C, mice were rechallenged with either 10e6 CD19+ or CD19neg Nalm6 on day 21 post-CAR-T infusion. Only the FOXO1-WT treated group mounted a response against the rechallenge, resulting in a significant survival advantage. 1-2 mice/CD19neg group and 2-5 mice/CD19+group. 1-way ANOVA and Dunnett's multiple comparisons test (FIG. 14A) and log-rank Mantel-Cox test (FIG. 14C) were used to determine statistical significance. * p<0.05; ** p<0.01; *** p<0.001

FIGS. 15A and 15B show data are derived from the experiment outlined in FIG. 13A. The graphs show the frequency of circulating CD4 and CD8 CAR-T cells from each time point as determined by flow cytometry. FOXO1-WT demonstrated the most robust expansion of CD8s (Day 7) compared to NGFR, TCF1, and FOXO1-3A groups, while both FOXO1-WT and FOXO1-3A demonstrated enhanced CD8 persistence compared to NGFR and TCF1 groups (days 15 and 21). n=3-7 mice/group from n=1 donor. 1-way ANOVA and Dunnett's multiple comparisons test was used to determine statistical significance. *** p<0.001; **** p<0.0001

FIG. 16A is graphs showing enhanced IFNy secretion and cytotoxicity in response to 143B tumor cells compared to controls for single (Her2.28z) or co-transduced (Her2.28z+FOXO1-WT) CAR-T cells purified on NGFR+ cells using magnetic bead selection. n=1 donor.

FIG. 16B is flow cytometry of co-transduced Her2.BBz CAR-T cells shows enhanced memory marker expression in NGFR+ (e.g., FOXO1-WT) cells vs NGFR- or Her2.BBz single transduced cells. FIG. 16C is a graph showing enhanced IL-2 secretion and cytotoxicity in response to 143B tumor cells compared to controls for single (Her2.BBz) or co-transduced (Her2.BBz+FOXO1-WT) CAR-T cells purified on NGFR+ cells using magnetic bead selection. n=1 donor. In FIGS. 16D and 16E, NSG mice were engrafted with 1e6 143B cells and infused with 5e6 Her2.BBz or Her2.BBz+FOXO1-WT CAR-T cells (of which approximately 50% were NGFR+). Caliper measurements of tumor volume show enhanced tumor control in mice treated with FOXO1-WT cells compared to those treated with Her2.BBz control cells. Data represents 4-5 mice/group from n=1 donor. 1-way ANOVA and Dunnett's multiple comparisons test was used to determine statistical significance. * p<0.05.

FIG. 17 shows transcription factor motif analysis of rested CAR-T cells demonstrating enhanced accessibility of memory associated transcription factor motifs compared to exhausted Always ON or non-exhausted Always OFF controls (Weber et al. 2021 Science).

FIGS. 18A-18C shows that ectopic FOXO1-WT reduces exhaustion when constitutively expressed in a bicistronic vector also containing the CAR sequence. FIG. 18A is a schematic of CAR T cells were retrovirally transduced with either HA.28z CAR alone (HA-28z) or a bicistronic vector containing both FOXO1-WT and the HA-28z CAR (FOXO1-HA-28z). FIG. 18B shows that FOXO1-HA-28z CAR-T cells display increased intracellular FOXO1, increased memory markers (CD62L and CD45RA) and decreased exhaustion markers (LAG-3 and TIM-3) compared to cells expressing only HA-28z. FIG. 18C are graphs showing that FOXO1-HA-28z CAR-T cells display enhanced cytokine secretion in response to tumor stimulation.

FIGS. 19A-19D show the characterization of CD19.BBQ CAR T cells overexpressing FOXO1-WT, FOXO1-3A, or an NGFR negative control serially challenged with Nalm6 leukemia cells at a 1:4 effector:target ratio on days 14, 17, 20, and 24 of culture. IFNγ secretion was measured after the fourth tumor challenge in two donors after 24 hours of CAR T cell-tumor co-culture (FIG. 19A). Data shows superior IFNγ secretion in cells engineered to overexpress FOXO1-WT compared to NGFR negative controls or cells that overexpress FOXO1-3A. Error bars show standard deviation in 3 separate wells. FIG. 19B is graphs of CD8+CAR T cell number after repeat tumor challenges. In both donors, CD8+cells that overexpressed FOXO1-WT expanded the most in response to serial tumor challenge. Cells were counted 3 days after tumor stimulation. FIG. 19C shows LEF1 expression in CAR T cells as measured by intracellular flow cytometry seven days after fourth tumor stimulation (n=2 independent donors). Cells that expressed FOXO1-WT had the most LEF1 expression which is correlated with memory-like phenotypes. FIG. 19D shows CD62L expression in CAR T cells as measured by surface flow cytometry seven days after fourth tumor stimulation (n=2 independent donors). Cells that expressed FOXO1-WT had the most CD62L expression which is correlated with memory-like phenotypes. Statistics in FIG. 19A were determined via two-tailed, unpaired student's T-test (***: p<0.0005, **: p<0.005).

FIGS. 20A and 20B show the metabolism of CD19.28(CAR T cells overexpressing FOXO1-WT, FOXO1-3A, or an NGFR negative control analyzed via Seahorse metabolic flux assay. FIG. 20A is a graph of the oxygen consumption rate (OCR) over time of CAR T cells. FOXO1-WT-overexpressing cells were the most metabolically active. Error bars show standard deviation of OCR measurement from at least 9 independent wells, n=1 donor. FIG. 20B is a graph of spare respiratory capacity (SRC) of CAR T cells as calculated by subtracting the OCR from timepoints (t) 7, 8, and 9 from timepoints 1, 2, and 3 in FIG. 20A. FOXO1-WT-overexpressing CAR T cells have the greatest spare respiratory capacity, indicating significantly healthier and more robust mitochondria. Bar height represents the average of the three SRC timepoint comparisons; error bars show standard deviation of SRC measurement from three timepoint comparisons (t7-t1, t8-t2, t9-t3; each timepoint OCR was determined by averaging the OCR values from 9 independent wells from that timepoint as in FIG. 20A). Statistics in FIG. 20B were determined via two-tailed, unpaired student's T-test (****: p<0.00005).

FIG. 21 a graph of additional results of tumor control and survival in mice treated with FOXO1-WT and FOXO1-3A CAR-T cells compared to those treated with untransduced (mock), NGFR, T cells, as in FIG. 12A. Nalm6 cells (1e6) were engrafted into NSG mice and a subtherapeutic dose of 0.2e6 CD19.28z+/NGFR+ T cells was infused 7 days post-engraftment. Data shows enhanced tumor control and survival in mice treated with FOXO1-WT compared to those treated with untransduced (mock), NGFR, or FOXO1-3A cells. 5 mice per group. 2 mice in the FOXO1-WT group were euthanized early due to complications that were unrelated to tumor progression. Statistics were determined using log-rank Mantel-Cox test.

### DETAILED DESCRIPTION

Herein, the expression and function of the master transcription factor FOXO1, wild-type (FOXO1-WT) or nuclear-restricted mutant FOXO1 (FOXO1-AAA), was leveraged to enhance CAR-T cell therapeutics. Ectopic expression of FOXO1 in human CAR-T cells endowed a more memory-like phenotype in vitro (based on cell surface markers) and enhanced killing in response to tumor in CAR-T cells targeting CD19 or Her2. These effects were irrespective of the co-stimulatory domain used in the CAR (e.g., CD28 vs 4-1BB). Further, using an in vitro T-cell exhaustion model in which a high-affinity GD2-targeting CAR spontaneously aggregates and tonically signals in the absence of antigen (HA-28z), ectopic expression of FOXO1 abrogated exhaustion and resulted in lower expression of inhibitory receptors, a more memory-like surface phenotype, and enhanced functionality in response to tumor.

Constitutive expression of FOXO1-WT in CAR-T cells resulted in enhanced tumor control in vivo using the following CAR-T/xenograft models: HA-28z versus GD2-expressing Nalm6 leukemia (liquid tumor); CD19-BBz versus Nalm6 leukemia (liquid tumor); and Her2-BBz versus 143B osteosarcoma (solid tumor)

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

As used herein, the terms “providing,” “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

) As used herein, “treat,” “treating” and the like means a slowing, stopping, or reversing of progression of a disease or disorder when provided a compound or composition described herein to an appropriate control subject. The term also means a reversing of the progression of such a disease or disorder to a point of eliminating or greatly reducing the symptoms. As such, “treating” means an application or administration of the compositions described herein to a subject, where the subject has a disease or a symptom of a disease, where the purpose is to cure, heal, alleviate, relieve, alter, remedy, ameliorate, improve, or affect the disease or symptoms of the disease.

A “vector” or “expression vector” is a replicon, such as plasmid, phage, virus, or cosmid, to which another DNA segment, e.g., an “insert,” may be attached or incorporated so as to bring about the replication of the attached segment in a cell.

### 2. COMPOSITIONS

Disclosed herein are compositions comprising isolated T cells that comprise an exogenous nucleic acid encoding a forkhead box protein O1 (FOXO1).

In some embodiments, the FOXO1 is wild-type FOXO1. In some embodiments, the FOXO1 comprises an amino acid sequence having at least about 70% (about 70%, about 75%, about 80%, about 85%, about 90%, about 95%, about 97%, about 98%, about 99%, or 100%) similarity to that of SEQ ID NO: 1. In some embodiments, the FOXO1 comprises an amino acid sequence of SEQ ID NO: 1

In some embodiments, the FOXO1 is a fully or partially nuclear restricted FOXO1 (e.g., FOXO1-AAA or FOXO1-3A, as disclosed herein). In some embodiments, the FOXO1 comprises an amino acid sequence having at least about 70% (about 70%, about 75%, about 80%, about 85%, about 90%, about 95%, about 97%, about 98%, about 99%, or 100%) similarity to that of SEQ ID NO: 2. In some embodiments, the FOXO1 comprises an amino acid sequence of SEQ ID NO: 2. In some embodiments, the FOXO1 is a not a nuclear restricted FOXO1. In some embodiments, the FOXO1 is partially nuclear restricted FOXO1, e.g., a variant of FOXO1 which has an increased likelihood of residing in the nucleus as compared to wild-type but is not entirely nuclear restricted. In some embodiments, the FOXO1 is not a partially nuclear restricted FOXO1.

SEQ ID NO: 1
MAEAPQVVEIDPDFEPLPRPRSCTWPLPRPEFSQSNSATSSPAPSGSAA
ANPDAAAGLPSASAAAVSADFMSNLSLLEESEDFPQAPGSVAAAVAAAA
AAAATGGLCGDFQGPEAGCLHPAPPQPPPPGPLSQHPPVPPAAAGPLAG
QPRKSSSSRRNAWGNLSYADLITKAIESSAEKRLTLSQIYEWMVKSVPY
FKDKGDSNSSAGWKNSIRHNLSLHSKFIRVQNEGTGKSSWWMLNPEGGK
SGKSPRRRAASMDNNSKFAKSRSRAAKKKASLQSGQEGAGDSPGSQFSK
WPASPGSHSNDDFDNWSTFRPRTSSNASTISGRLSPIMTEQDDLGEGDV
HSMVYPPSAAKMASTLPSLSEISNPENMENLLDNLNLLSSPTSLTVSTQ
SSPGTMMQQTPCYSFAPPNTSLNSPSPNYQKYTYGQSSMSPLPQMPIQT
LQDNKSSYGGMSQYNCAPGLLKELLTSDSPPHNDIMTPVDPGVAQPNSR
VLGQNVMMGPNSVMSTYGSQASHNKMMNPSSHTHPGHAQQTSAVNGRPL
PHTVSTMPHTSGMNRLTQVKTPVQVPLPHPMQMSALGGYSSVSSCNGYG
RMGLLHQEKLPSDLDGMFIERLDCDMESIIRNDLMDGDTLDFNFDNVLP
NQSFPHSVKTTTHSWVSG
SEQ ID NO: 2
MAEAPQVVEIDPDFEPLPRPRSCAWPLPRPEFSQSNSATSSPAPSGSAA
ANPDAAAGLPSASAAAVSADFMSNLSLLEESEDFPQAPGSVAAAVAAAA
AAAATGGLCGDFQGPEAGCLHPAPPQPPPPGPLSQHPPVPPAAAGPLAG
QPRKSSSSRRNAWGNLSYADLITKAIESSAEKRLTLSQIYEWMVKSVPY
FKDKGDSNSSAGWKNSIRHNLSLHSKFIRVQNEGTGKSSWWMLNPEGGK
SGKSPRRRAAAMDNNSKFAKSRSRAAKKKASLQSGQEGAGDSPGSQFSK
WPASPGSHSNDDFDNWSTFRPRTSANASTISGRLSPIMTEQDDLGEGDV
HSMVYPPSAAKMASTLPSLSEISNPENMENLLDNLNLLSSPTSLTVSTQ
SSPGTMMQQTPCYSFAPPNTSLNSPSPNYQKYTYGQSSMSPLPQMPIQT
LQDNKSSYGGMSQYNCAPGLLKELLTSDSPPHNDIMTPVDPGVAQPNSR
VLGQNVMMGPNSVMSTYGSQASHNKMMNPSSHTHPGHAQQTSAVNGRPL
PHTVSTMPHTSGMNRLTQVKTPVQVPLPHPMQMSALGGYSSVSSCNGYG
RMGLLHQEKLPSDLDGMFIERLDCDMESIIRNDLMDGDTLDFNFDNVLP
NQSFPHSVKTTTHSWVSG

A FOXO1 suitable for the disclosed compositions and methods may comprise one or more amino acid substitutions or truncations as compared to the corresponding wild-type protein or SEQ ID NO: 1. In some embodiments, the FOXO1 is a functional fragment of wild-type FOXO1 or SEQ ID NO: 1. In some embodiments, the FOXO1 is a functional variant of wild-type FOXO1 or SEQ ID NO: 1, such that the primary amino acid sequence may contain one or more substitutions while the resulting polypeptide retains its expression levels, cellular localization and/or activities (e.g., promoting and maintaining CAR-T cells memory phenotypes and/or upregulation of transcription factors known to drive stemness and effector transcriptional programs). The functional variant preferably retains greater than 50% of the activity (e.g., prevention of T cell exhaustion) of the original polypeptide.

An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH2 can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

The exogenous nucleic acid facilitates overexpression of FOXO1 in the T cells. The expression of FOXO1 may be constitutive, regulatable or inducible, cell type specific, tissue-specific, or species specific. In select embodiments, the isolated T cells constitutively express FOXO1 from the exogenous nucleic acid.

In some embodiments, the T cells maintain functionality under conditions in which unmodified T cells, T cells not expressing FOXO1 from an exogenous nucleic acid, display exhaustion (e.g., maintaining functionality of T cells exposed to excessive antigen). “T cell exhaustion” refers to loss of T cell function, which may occur as a result of an infection (e.g., a chronic infection) or a disease. T cell exhaustion is associated with increased expression of exhaustion markers and inhibitory receptors (e.g., PD-1, TIM-3, and LAG-3), apoptosis, and reduced cytokine secretion.

In some embodiments, FOXO1 is fused to a motif which modulates expression levels or enhances intracellular degradation (e.g., degron, destabilizing domain, or the like). The motif may be fused to the N-terminus or C-terminus of FOXO1. The motif may be attached via a linker to the FOXO1.

In some embodiments, FOXO1 is fused to a destabilizing domain (DD). A destabilizing domain is a protein domain which modulates the stabilization of a payload (e.g., a protein of interest (e.g., FOXO1)) fused to the DD as a result of the absence or presence of a binding ligand (e.g., small molecule or drug). For example, some destabilizing domains in the absence of its binding ligand result in recognition and degradation of the payload fused to the DD by the ubiquitin-proteasome system. While in the presence of its binding ligand, the fused DD and payload are stabilized. In some instances, the stability is dose dependent. Thus, the presence, absence or an amount of a small molecule ligand that binds to or interacts with the DD, can, upon such binding or interaction modulate the stability of the FOXO1 and consequently the function of FOXO1. Accordingly, the presence of a tunable destabilizing domain allows the concentration of the FOXO1 to be modulated over time with the cognate binding ligand.

In some embodiments, FOXO1 is fused to a degron or one or more degrons. As used herein, a “degron” is a single amino acid or peptide capable of targeting the FOXO1 for degradation. Any suitable degron may be used as is deemed appropriate for an intended use based on the disclosure herein. Degrons include portions of proteins that signal and/or target for degradation (or otherwise increase the degradation rate of) the protein to which the degron is attached or otherwise associated (e.g., grafted onto). Non-limiting examples of degrons include short amino acid sequences, structural motifs, exposed amino acids, and the like. Degrons may be prokaryote or eukaryote derived and may be employed in naturally occurring or non-naturally occurring (i.e., recombinant) forms. Degrons may be post-translationally modified to target a protein for degradation where such post-translational modifications include but are not limited to e.g., ubiquitination, proteolytic cleavage, phosphorylation, methylation, ADP-ribosylation, AMPylation, lipidation, alkylation, nitrosylation, succinylation, SUMOylation, neddylation, ISGylation, and the like. Useful degrons include ubiquitin-dependent degrons and ubiquitin-independent degrons. For example, in some instances, a protein may be targeted for ubiquitin-independent proteasomal degradation by attachment of an ornithine decarboxylase (ODC) degron, including but not limited to e.g., a mammalian ODC such as e.g., a rodent ODC, including but not limited to e.g., the c-terminal mouse ODC (cODC). In some instances, useful degrons include those described in Takeuchi et al., Biochem. J (2008) 410:401-407 and/or Matsuzawa et al., PNAS (2005) 102(42):14982-7; the disclosures of which are incorporated herein by reference in their entirety. In some instances, a protein may be targeted for ubiquitin-independent proteasomal degradation by post-translational modification (including but not limited to e.g., proteolytic cleavage, phosphorylation, methylation, ADP-ribosylation, AMPylation, lipidation, alkylation, nitrosylation, succinylation, SUMOylation, neddylation, ISGylation) of a degron, where such modification leads, directly or indirectly, to partial or complete unfolding of the protein or other mechanisms that lead to degradation of the protein.

The invention is not limited by the type of T cell modified to overexpress and/or comprise an exogenous nucleic acid molecule encoding FOXO1. The T cells may be selected from CD3+ T cells, CD8+ T cells, CD4+ T cells, natural killer (NK) T cells, alpha beta T cells, gamma delta T cells, or any combination thereof (e.g., a combination of CD4+ and CD8+ T cells). In some embodiments, the T cells are memory T cells (e.g., central memory T cells or effector memory T cells). In some embodiments, the T cells are tumor infiltrating lymphocytes. In some embodiments, the T cells are cytokine-induced killer cells.

In some embodiments, the T cells are naturally occurring T cells. For example, the T cells may be isolated from a subject sample. In some embodiments, the T cell is an anti-tumor T cell (e.g., a T cell with activity against a tumor (e.g., an autologous tumor) that becomes activated and expands in response to antigen). Anti-tumor T cells include, but are not limited to, T cells obtained from resected tumors or tumor biopsies (e.g., tumor infiltrating lymphocytes (TILs)) and a polyclonal or monoclonal tumor-reactive T cell (e.g., obtained by apheresis, expanded ex vivo against tumor antigens presented by autologous or artificial antigen-presenting cells). In some embodiments, the T cells are expanded ex vivo.

In some embodiments, the isolated T cells further comprise a nucleic acid encoding a recombinant receptor. In some embodiments, the recombinant receptor is a T cell receptor (TCR) or a chimeric antigen receptor (CAR).

In certain embodiments, the T cells are genetically modified with recombinant receptors that recognize and respond to tumor antigens. Such receptors are generally composed of extracellular domains comprising a single-chain antibody (scFv) specific for tumor antigen, linked to intracellular T cell signaling motifs (See, e.g., Westwood, J. A. et al, 2005, Proc. Natl. Acad. Sci., USA, 102(52):19051-19056).

The invention is not limited by the type of tumor antigen recognized. The term “tumor antigen” as used herein refers to any molecule (e.g., protein, peptide, lipid, carbohydrate, etc.) solely or predominantly expressed or over-expressed by a tumor cell or cancer cell, such that the antigen is associated with the tumor or cancer. The cancer antigen can additionally be expressed by normal, non-tumor, or non-cancerous cells. However, in such cases, the expression of the cancer antigen by normal, non-tumor, or noncancerous cells is not as robust as the expression by tumor or cancer cells. In this regard, the tumor or cancer cells can over-express the antigen or express the antigen at a significantly higher level, as compared to the expression of the antigen by normal, non-tumor, or noncancerous cells. Also, the cancer antigen can additionally be expressed by cells of a different state of development or maturation. For instance, the cancer antigen can be additionally expressed by cells of the embryonic or fetal stage, which cells are not normally found in an adult. Alternatively, the cancer antigen can be additionally expressed by stem cells or precursor cells, which cells are not normally found in an adult.

The cancer antigen can be an antigen expressed by any cell of any cancer or tumor. The cancer antigen may be a cancer antigen of only one type of cancer or tumor, such that the cancer antigen is associated with or characteristic of only one type of cancer or tumor. Alternatively, the cancer antigen may be a cancer antigen (e.g., may be characteristic) of more than one type of cancer or tumor. For example, the cancer antigen may be expressed by both breast and prostate cancer cells and not expressed at all by normal, non-tumor, or non-cancer cells. Exemplary cancer antigens include, but are not limited to, glycoprotein 100 (gp100), melanoma antigen recognized by T cells 1 (MART-1), melanoma antigen gene (MAGE) Family Members (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12), New York esophageal squamous cell carcinoma 1 (NY-ESO-1), vascular endothelial growth factor receptor-2 (VEGFR-2), glioma-associated antigen, carcinoembryonic antigen (CEA), beta-human chorionic gonadotropin, alphafetoprotein (AFP), lectin-reactive AFP, thyroglobulin, human telomerase reverse transcriptase, prostate-specific antigen (PSA), prostate-carcinoma tumor antigen-1 (PCTA-1), insulin growth factor (IGF)-I, IGF-II, IGF-I receptor, intestinal carboxyl esterase, human epidermal growth factor receptor 2 (HER-2), mesothelin, and epidermal growth factor receptor variant III (EGFR III).

Any T cell containing a receptor that recognizes a tumor antigen finds use in the compositions and methods of the invention. Examples include, but are not limited to, T cells expressing a receptor (e.g., a native or naturally occurring receptor, or a receptor engineered to express a synthetic receptor such as an engineered TCR or a CAR) that recognize an antigen selected from CD19, CD20, CD22, receptor tyrosine kinase-like orphan receptor 1 (RORI), disialoganglioside 2 (GD2), Epstein-Barr Virus (EBV) protein or antigen, folate receptor, mesothelin, human carcinoembryonic antigen (CEA), prostatic acid phosphatase (PAP), CD33/IL3R, tyrosine protein kinase Met (c-Met) or hepatocyte growth factor receptor (HGFR), prostate-specific membrane antigen (PSMA), Glycolipid F77, epidermal growth factor receptor variant III (EGFRvIII), NY-ESO-1, melanoma antigen gene (MAGE) Family Member A3 (MAGE-A3), melanoma antigen recognized by T cells 1 (MART-1), GP1000, p53, or other tumor antigen described herein.

In some embodiments, the T cell is engineered to express a chimeric antigen receptor (CAR). Any CAR that binds with specificity to a desired antigen (e.g., tumor antigen) may be utilized with the present invention. In certain embodiments, the CAR comprises an antigen-binding domain. In certain embodiments, the antigen-binding domain is a single-chain variable fragment (scFv) containing heavy and light chain variable regions that bind with specificity to the desired antigen. In some embodiments, the CAR further comprises a transmembrane domain (e.g., a T cell transmembrane domain (e.g., a CD28 transmembrane domain)) and a signaling domain comprising one or more immunoreceptor tyrosine-based activation motifs (ITAMs)(e.g., a T cell receptor signaling domain (e.g., TCR zeta chain)). In some embodiments, the CAR comprises one or more co-stimulatory domains (e.g., domains that provide a second signal to stimulate T cell activation). The invention is not limited by the type of co-stimulatory domain. Indeed, any co-stimulatory domain known in the art may be used including, but not limited to, CD28, OX40/CD134, 4-1BB/CD137/TNFRSF9, the high affinity immunoglobulin E receptor-gamma subunit (FcERIγ, ICOS/CD278, interleukin 2 subunit beta (ILRβ) or CD122, cytokine receptor common subunit gamma (IL-2Rγ) or CD132, and CD40. In some embodiments, the co-stimulatory domain is 4-1BB. In some embodiments, the co-stimulatory domain is CD28.

The CAR may comprise a target-specific binding element otherwise referred to as an antigen binding moiety. The choice of moiety depends upon the type and number of ligands that define the surface of a target cell. For example, the antigen binding domain may be chosen to recognize a ligand that acts as a cell surface marker on target cells associated with a particular disease state. Examples of cell surface markers that may act as ligands for the antigen moiety domain in the CAR of the invention include those associated with viral, bacterial and parasitic infections, autoimmune disease and, as described above, cancer cells.

Depending on the desired antigen to be targeted, a CAR can be engineered to include the appropriate antigen binding moiety specific to the desired antigen target. For example, if CD19 is the desired antigen that is to be targeted, an antibody for CD19 can be used as the antigen binding moiety for incorporation into the CAR of the invention.

FOXO1 and the engineered receptor may be encoded by the same or different nucleic acids. In some embodiments, FOXO1 and the engineered receptor are encoded by a single nucleic acid. In some embodiments, FOXO1 and the engineered receptor are encoded by separate nucleic acids. The nucleic acid(s) may comprise DNA or RNA (e.g., mRNA). In some embodiments, the nucleic acid(s) comprise vectors.

FOXO1 and the engineered receptor may be expressed using the same, similar (e.g., both weak or both strong), or different promoters. For example, the promoter for FOXO1 may confer a high rate of transcription (a strong promoter), whereas the promoter for the engineered receptor may confer a low rate of transcription (weak promoter), or vice versa. In some embodiments, the promoter for FOXO1 and the engineered receptor may confer a high rate of transcription (a strong promoter). In some embodiments, the promoter for FOXO1 and the engineered receptor may confer a low rate of transcription (weak promoter). Many promoter libraries have been established experimentally and choice of promoter and promoter strength is well-known to one of skill in the art.

Further, the nucleic acids of the present disclosure may comprise a promoter that is constitutive, regulatable or inducible, cell type specific, tissue-specific, or species specific. In addition to the sequence sufficient to direct transcription, the promoter may also include sequences of other regulatory elements that are involved in modulating transcription (e.g., enhancers, Kozak sequences and introns). Many promoter/regulatory sequences useful for driving constitutive expression are available in the art and include, but are not limited to, for example, CMV (cytomegalovirus promoter), EF1 a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible expression can be accomplished by placing the nucleic acid encoding such a molecule under the control of an inducible promoter/regulatory sequence. Promoters that are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The present disclosure also provides for vectors containing the nucleic acids and cells containing the nucleic acids or vectors, thereof. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

Expression vectors for stable or transient expression may be constructed via conventional methods and introduced into cells. For example, nucleic acids may be cloned into a suitable expression vector, such as a plasmid or a viral vector in operable linkage to a suitable promoter. The selection of expression vectors/plasmids/viral vectors are preferably suitable for integration and replication in eukaryotic cells.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Additionally, the vector may contain, for example, some or all of the following: a selectable marker gene for selection of stable or transient transfectants in host cells; transcription termination and RNA processing signals; 5′- and 3′-untranslated regions; internal ribosome binding sites (IRESes), versatile multiple cloning sites; and reporter gene for assessing expression of the chimeric receptor. Suitable vectors and methods for producing vectors containing transgenes are well known and available in the art. Selectable markers include chloramphenicol resistance, tetracycline resistance, spectinomycin resistance, neomycin, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into a cell, the vectors may be maintained as an autonomously replicating sequence or extrachromosomal element or may be integrated into host DNA. The nucleic acids may be delivered to the cells by any suitable means.

Conventional viral and non-viral based gene transfer methods can be used to introduce the nucleic acids into cells. Such methods can be used to administer the nucleic acids to cells in culture, or in a host organism. Non-viral vector delivery systems include DNA plasmids, cosmids, RNA (e.g., a transcript of a vector described herein), a nucleic acid, and a nucleic acid complexed with a delivery vehicle.

Viral vector delivery systems include DNA and RNA viruses, which have either episomal or integrated genomes after delivery to the cell. A variety of viral constructs may be used to deliver the present nucleic acids to the cells. Viral vectors include, for example, retroviral, lentiviral, adenoviral, adeno-associated and herpes simplex viral vectors. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

Vectors according to the present disclosure can be transformed, transfected, or otherwise introduced into cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment).

Additionally, delivery vehicles such as nanoparticle- and lipid-based delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1; 459(1-2):70-83), incorporated herein by reference.

The composition may optionally include at least one additional therapeutic agent, such as other drugs for treating T cell exhaustion (e.g., anti-PD-1 checkpoint inhibitor, such as nivolumab), or other medications used to treat a subject for an infection or disease associated with T cell exhaustion (e.g., antiviral, antibiotic, antimicrobial, or anti-cancer drugs).

In some embodiments, the at least one additional therapeutic agent comprises at least one chemotherapeutic agent. As used herein, the term “chemotherapeutic” or “anti-cancer drug” includes any small molecule or other drug used in cancer treatment or prevention. Chemotherapeutics include, but are not limited to, cyclophosphamide, methotrexate, 5-fluorouracil, doxorubicin, docetaxel, daunorubicin, bleomycin, vinblastine, dacarbazine, cisplatin, paclitaxel, raloxifene hydrochloride, tamoxifen citrate, abemacicilib, afinitor (Everolimus), alpelisib, anastrozole, pamidronate, anastrozole, exemestane, capecitabine, epirubicin hydrochloride, eribulin mesylate, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, and tucatinib. In select embodiments, the chemotherapeutic agent comprises paclitaxel.

The compositions can include, for example, cytokines, chemokines and other biologic signaling molecules, tumor specific vaccines, cellular cancer vaccines (e.g., GM-CSF transduced cancer cells), tumor specific monoclonal antibodies, autologous and allogeneic stem cell rescue (e.g., to augment graft versus tumor effects), other therapeutic antibodies, molecular targeted therapies, anti-angiogenic therapy, infectious agents with therapeutic intent (such as tumor localizing bacteria) and gene therapy.

The compositions may include pharmaceutically acceptable carriers. The term “pharmaceutically acceptable carrier,” as used herein, means a non-toxic, inert solid, semi-solid or liquid filler, diluent, encapsulating material, surfactant, cyclodextrins or formulation auxiliary of any type. A carrier may include a single ingredient or a combination of two or more ingredients. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; cyclodextrins such as, but not limited to, alpha-CD, beta-CD, gamma-CD, HP-beta-CD, SBE-beta-CD; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, preservatives and antioxidants can also be present in the composition, according to the judgment of the formulator.

The route of administration and the form of the composition will dictate the type of carrier to be used. The composition may be in a variety of forms, suitable, for example, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis).

### 3. METHODS

The present disclosure also provides methods for treating a disease or disorder. In some embodiments, the methods comprise administering to the subject an effective amount of T cells modified to express and/or contain elevated levels of FOXO1. In some embodiments, the methods comprise administering to the subject an effective amount of a composition as described herein. The invention is not limited by the type of disease or condition treated. Any disease or condition that is treatable via administration of T cells can be treated in an improved and more effective manner using compositions and methods of the invention (e.g., containing and/or using T cells modified to express and/or contain elevated levels of FOXO1).

In some embodiments, the administration inhibits or reduces T cell exhaustion (e.g., as compared to a subject receiving the same amount of engineered T cells (e.g., CAR T cells or T cells comprising a recombinant TCR) not modified to express and/or contain elevated levels of FOXO1). In some embodiments, the administration results in lower expression of inhibitory receptors (e.g., programed cell death 1 (PDCD1, also called PD1) and cytotoxic T lymphocyte-associated Antigen 4 (CTLA-4)) compared to the administration of unmodified T cells.

In some embodiments, the exogenous FOXO1 is expressed in the T cells prior to introduction of the recombinant receptor to the T cells. In some embodiments, the exogenous FOXO1 and the recombinant receptor are introduced to the T cells at the same time and expression is simultaneous or substantially simultaneous. In some embodiments, the recombinant receptor is expressed in the T cells prior to introduction of the exogenous FOXO1 to the T cells.

In some embodiments, the FOXO1 is overexpressed in the T cells prior to exposure to the antigen.

The T cells may be isolated from a subject. In some embodiments, the T cells are allogeneic to the subject. In some embodiments, the T cells are autologous to the subject. Thus, the T cells may be isolated from a sample from the subject, modified and expanded ex vivo, and returned to the subject.

In some embodiments, the disease or condition is cancer. In some embodiments, the disease or condition is an infectious disease. The invention is not limited by the type of cancer or by the type of infectious disease. Indeed, any cancer known in the art for which T cell therapy is used for treatment may be treated with the compositions and methods of the invention. In like manner, any infectious disease known in the art for which T cell therapy is used for treatment may be treated with the compositions and methods of the invention.

In certain embodiments, the invention provides methods for treating or delaying the progression of cancer, or for treating or delaying the progress of infectious disease, in an individual comprising administering to the individual an effective amount of modified T cells or compositions thereof, as described herein. In some embodiments, the treatment results in a sustained response in the individual after cessation of the treatment.

The methods can be used with any cancer cell or in a subject having any type of cancer, for example those described by the National Cancer Institute. In some embodiments, the cancer may be a carcinoma, sarcoma, lymphoma, leukemia, melanoma, mesothelioma, multiple myeloma, or seminoma. In some embodiments, the cancer comprises a solid tumor. In some embodiments, the cancer is metastatic cancer.

The cancer may be a cancer of the bladder, blood, bone, brain, breast, cervix, colon/rectum, endometrium, head and neck, kidney, liver, lung, muscle tissue, ovary, pancreas, prostate, skin, spleen, stomach, testicle, thyroid, or uterus.

The methods described herein may find use in treating conditions where enhanced immunogenicity is desired such as increasing tumor immunogenicity for the treatment of cancer. In some embodiments, the recombinant receptor (e.g., CAR and/or TCR) is specific for the cancer being treated. In some embodiments, the recombinant receptor (e.g., CAR and/or TCR) is generic for all cancers.

In certain embodiments, the present invention demonstrates that treatment of a subject having cancer with a therapeutically effective amount of the disclosed compositions is superior to treatment of a subject having cancer with unmodified T cells. In some embodiments, treatment with therapeutically effective amounts of the disclosed compositions inhibits the development or growth of cancer cells or and/or renders the cancer cells as a population more susceptible to other treatments (e.g., the cell death-inducing activity of cancer therapeutic drugs or radiation therapies). Accordingly, compositions and methods of the invention may be used as a monotherapy (e.g., to kill cancer cells, and/or reduce or inhibit cancer cell growth, induce apoptosis and/or cell cycle arrest in cancer cells), or when administered in combination with one or more additional agent(s), such as other anti-cancer agents (e.g., cell death-inducing or cell cycle-disrupting cancer therapeutic drugs or radiation therapies) to render a greater proportion of the cancer cells susceptible to killing, inhibited cancer cell growth, induced apoptosis and/or cell cycle arrest compared to the corresponding proportion of cells in an animal treated only with the cancer therapeutic drug or radiation therapy alone.

In some embodiments, the individual has cancer that is resistant (e.g., has been demonstrated to be resistant) to one or more other forms of anti-cancer treatment (e.g., chemotherapy, immunotherapy, etc.). In some embodiments, resistance includes recurrence of cancer or refractory cancer. Recurrence may refer to the reappearance of cancer, in the original site or a new site, after treatment. In some embodiments, resistance includes progression of the cancer during treatment with chemotherapy. In some embodiments, resistance includes cancer that does not respond to traditional or conventional treatment with a chemotherapeutic agent. The cancer may be resistant at the beginning of treatment or it may become resistant during treatment. In some embodiments, the cancer is at early stage or at late stage.

In some embodiments, the modified T cells and compositions thereof are used to treat, ameliorate, or prevent a cancer that is characterized by resistance to one or more conventional cancer therapies (e.g., those cancer cells which are chemoresistant, radiation resistant, hormone resistant, and the like). In some embodiments, the treatment may inhibit the growth of resistant cancer cells outright and/or render such cells as a population more susceptible to cancer therapeutic drugs or radiation therapies (e.g., to the cell death-inducing activity thereof).

In certain embodiments, the therapeutically effective amount of the modified T cell composition reduces the number of cancer cells in the patient following such treatment. In certain embodiments, the therapeutically effective amount of the modified T cell composition reduces and/or eliminates the tumor burden in the patient following such treatment.

A wide range of second therapies may be used in conjunction with the methods of the present disclosure. The second therapy may be administration of an additional therapeutic agent or may be a second therapy not connected to administration of another agent. Such second therapies include, but are not limited to, surgery, immunotherapy, radiotherapy, or an additional chemotherapeutic or anti-cancer agent.

The second therapy may be administered at the same time as the initial therapy, either in the same composition or in a separate composition administered at substantially the same time as the first composition. In some embodiments, the second therapy may precede or follow the treatment of the first therapy by time intervals ranging from hours to months.

In certain embodiments, the method further comprises administering radiation therapy to the patient. In certain embodiments, the radiation therapy is administered before, at the same time as, and/or after the patient receives the therapeutically effective amount of the modified T cell composition.

In certain embodiments, the method further comprises administering to the patient one or more anticancer agents and/or one or more chemotherapeutic agents. In certain embodiments, the one or more anticancer agents and/or one or more chemotherapeutic agents are administered before, at the same time as, and/or after the patient receives the therapeutically effective amount of the modified T cell composition. In certain embodiments, combination treatment of a patient with a therapeutically effective amount of modified T cells and a course of an anticancer agent produces a greater tumor response and clinical benefit in such patient compared to those treated with the modified T cells or anticancer drugs/radiation alone. Since the doses for all approved anticancer drugs and radiation treatments are known, the present invention contemplates the various combinations of them with the modified T cells.

In some embodiments, the second therapy comprises administration of antibodies. The antibodies may target antigens either specifically expressed by tumor cells or antigens shared with normal cells. In some embodiments, the antibody may target, for example, CD20, CD33, CD52, CD30, HER (also referred to as erbB or EGFR), VEGF, CTLA-4 (also referred to as CD152), epithelial cell adhesion molecule (EpCAM, also referred to as CD326), and PD-1/PD-L1. Suitable antibodies include, but are not limited to, rituximab, blinatumomab, trastuzumab, gemtuzumab, alemtuzumab, ibritumomab, tositumomab, bevacizumab, cetuximab, panitumumab, ofatumumab, ipilimumab, brentuximab, pertuzumab and the like). In some embodiments, the additional therapeutic agent may comprise anti-PD-1/PD-L1 antibodies, including, but not limited to, pembrolizumab, nivolumab, cemiplimab, atezolizumab, avelumab, durvalumab, and ipilimumab. The antibodies may also be linked to a chemotherapeutic agent. Thus, in some embodiments, the antibody is an antibody-drug conjugate.

The administration of second therapy may be administered to a subject by a variety of methods. In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof.

The present disclosure also provides methods preventing exhaustion (e.g., maintaining functionality of T cells exposed to excessive antigen) of engineered T cells comprising introducing a nucleic acid that overexpresses FOXO1 into the T cells. In some embodiments the methods further comprise administering the engineered T cells to a subject in need thereof.

“Preventing T cell exhaustion” refers to a condition of restored functionality of T cells characterized by one or more of the following: decreased expression and/or level of one or more of PD-1, TIM-3, and LAG-3; increased memory cell formation and/or maintenance of memory markers (e.g., CD62L); prevention of apoptosis; increased antigen-induced cytokine (e.g., IL-2) production and/or secretion; enhanced killing capacity; increased recognition of tumor targets with low surface antigen; enhanced proliferation in response to antigen; and lower expression of inhibitory receptors (e.g., programed cell death 1 (PDCD1, also called PD1) and cytotoxic T lymphocyte-associated Antigen 4 (CTLA-4)).

Accordingly, the modified T cells may display increased functionality and/or activity (e.g., increased antigen induced cytokine production, enhanced killing capacity (e.g., increased recognition of tumor targets with low surface antigen), increased memory cell formation, and/or enhanced proliferation in response to antigen) and/or reduced features of exhaustion (e.g., lower levels of markers or inhibitory receptors indicative of exhaustion (e.g., PD-1, TIM-3, LAG-3) and/or lower levels of programmed cell death) compared to non-modified T cells. In the context of therapeutic applications, the modified T cells may enhance the clinical efficacy of the therapeutics (e.g., CAR T cells).

In some embodiments, the isolated T cells further comprise a nucleic acid encoding a recombinant receptor. In some embodiments, the recombinant receptor is a T cell receptor (TCR) or a chimeric antigen receptor (CAR). In some embodiments, the recombinant receptor is specific for a tumor antigen.

Descriptions of the FOXO1, the recombinant receptor, the nucleic acids and target antigens thereof, the subject, and the disease and disorders set forth above in connection with the inventive compositions are also applicable to the method of preventing exhaustion of engineered T cells.

An effective amount of the modified T cells or compositions disclosed herein may be determined based on the type of disease to be treated, the type of modified T cell, the severity and course of the disease, the clinical condition of the individual, the individual's clinical history and response to the treatment, and the discretion of the attending physician.

The efficacy of any of the methods described herein (e.g., treatment of disease or disorder) may be tested in various models known in the art, such as clinical or pre-clinical models. Effectiveness of the treatment may refer to any one or more of: extending survival (including overall survival and progression free survival); resulting in an objective response (including a complete response or a partial response); or improving signs or symptoms of the disease or disorder (e.g., cancer or an infection disease).

In some embodiments, a sample is obtained prior to treatment with T cells (e.g., alone or in combination with another therapy described herein) as a baseline for measuring response to treatment. In some embodiments, the sample is a tissue sample (e.g., formalin-fixed and paraffin-embedded (FFPE), archival, fresh or frozen). In some embodiments, the sample is whole blood. In some embodiments, the whole blood comprises immune cells, circulating tumor cells and any combinations thereof.

For any exemplary cancer model, after developing tumors, mice may be placed into treatment groups receiving treatment or control treatment. Tumor size (e.g., tumor volume) is measured during the course of treatment, and overall survival rate is also monitored.

In some embodiments, efficacy may refer to improvement of one or more factors according to the published set of RECIST guidelines for determining the status of a tumor in a cancer patient, i.e., responding, stabilizing, or progressing. A responsive subject may refer to a subject whose cancer(s) show improvement, e.g., according to one or more factors based on RECIST criteria. A non-responsive subject may refer to a subject whose cancer(s) do not show improvement, e.g., according to one or more factors based on RECIST criteria.

Effectiveness may also refer to improvement of one of more immune-related response criteria (irRC). In some embodiments, new lesions are added into the defined tumor burden and followed, e.g., for radiological progression at a subsequent assessment. In some embodiments, presence of non-target lesions is included in assessment of complete response and not included in assessment of radiological progression. In some embodiments, radiological progression may be determined only on the basis of measurable disease and/or may be confirmed by a consecutive assessment >4 weeks from the date first documented.

The disclosure further provides kits containing one or more reagents or other components useful, necessary, or sufficient for practicing any of the methods described herein. For example, kits may include FOXO1 reagents (nucleic acids, vectors, compositions, etc.), recombinant vector reagents (nucleic acids, vectors, compositions, etc.), transfection or administration reagents, negative and positive control samples (e.g., T cells or empty vector DNA), T cells, containers (e.g., microcentrifuge tubes), detection and analysis instruments, software, instructions, and the like.

### 4. EXAMPLES

### Example 1

### CAR T Cell Exhaustion

CARs are synthetic proteins that most commonly combine an extracellular tumor recognition domain with intracellular domains that coopt T cell receptor (TCR) machinery and enable T cells to recognize and destroy tumor cells in a major histocompatibility complex-independent manner. CAR T cells have exhibited unprecedented response rates in hematological malignancies. However, T cell exhaustion, a process whereby CD8+ T cells experiencing chronic antigen stimulation through the TCR/CAR progressively lose effector function, has impeded their efficacy in liquid and solid tumors. Targeting exhaustion via checkpoint blockade, while effective in some patients, does not remodel the epigenetic imprint of exhaustion, convert exhausted cells into memory cells, or improve the CAR T cell efficacy, underscoring the need for alternative approaches.

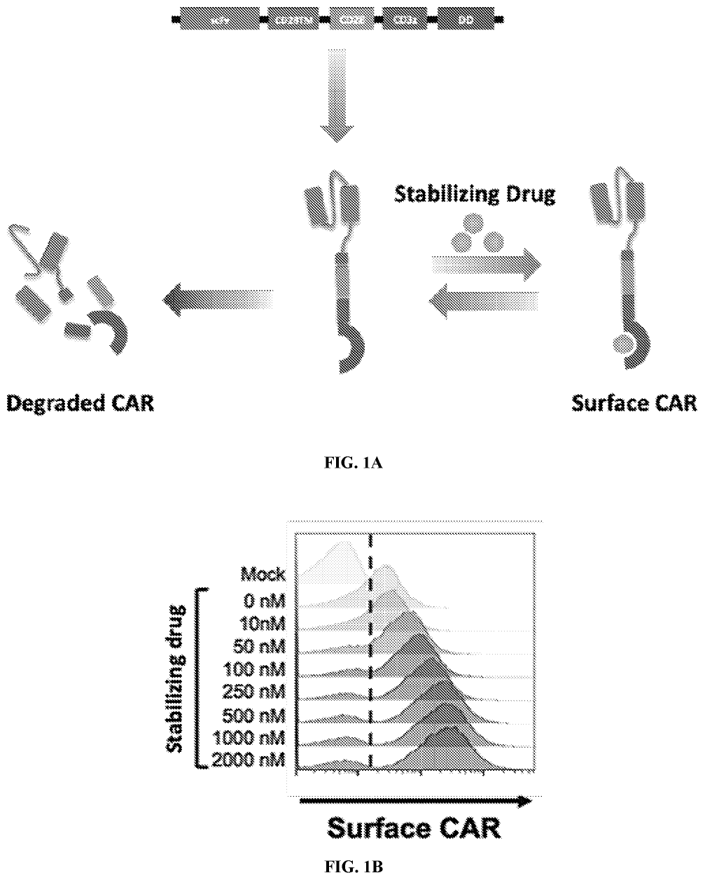

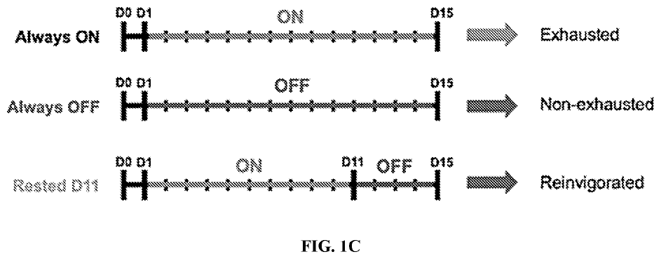

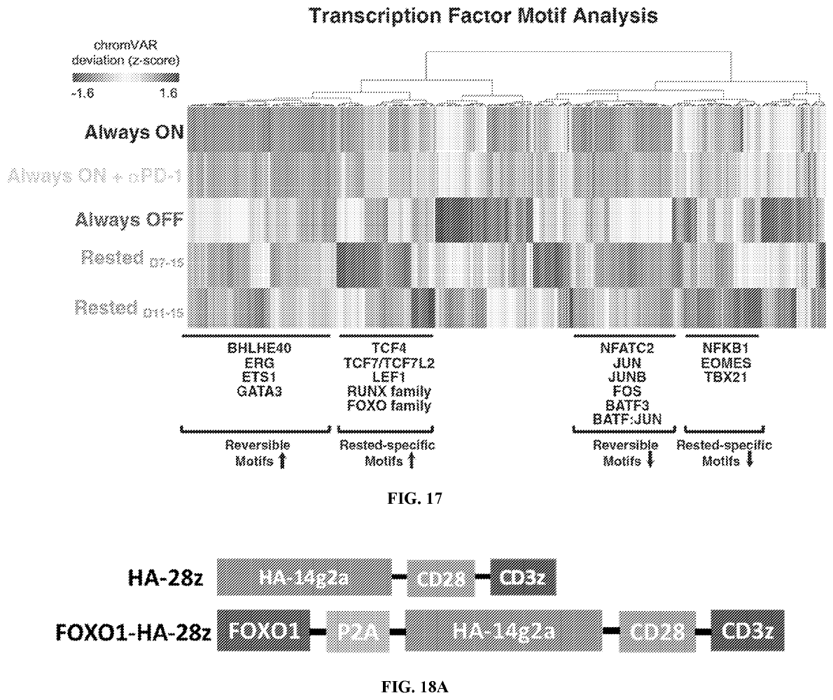

A novel model was created in which T cells express a high-affinity GD2-targeting CAR (HA-28z) that tonically signals in the absence of antigen due to spontaneous CAR aggregation, thus simulating persistent antigen exposure. HA-28z CAR T cells rapidly acquire phenotypic, functional, transcriptional, and epigenetic hallmarks of exhaustion. To test if transient disruption of CAR signaling would functionally reinvigorate exhausted CAR T cells and promote memory, the HA-28z CAR was modified with a C-terminal destabilizing domain (DD), enabling drug-dependent control of CAR protein, anti-tumor function, and tonic signaling (FIGS. 1A-1B). Transient downregulation of HA-28z CAR and cessation of tonic CAR signaling, or “rest” (FIG. 1C), reversed dysfunction, diminished inhibitory receptor expression, and promoted a memory-like phenotype. Rest also induced global transcriptional and epigenetic reprogramming of exhausted CAR T cells, resulting in reversion to a state that more closely resembles healthy, memory-like CAR T cells. Epigenetic changes included increased accessibility of motifs bound by memory-associated transcription factors in rested cells (ex. FOXO family transcription factors, TCF7) (FIG. 17), thereby implicating the activity of these in the factors in the reversal or mitigation of exhaustion. Reversal of dysfunction mediated by rest is dependent on EZH2 activity, suggesting a causal relationship between epigenetic reprogramming and enhanced CAR T cell functionality.

### Example 2

### Endogenous FOXO1 for CAR-T Cell Survival and Memory Formation

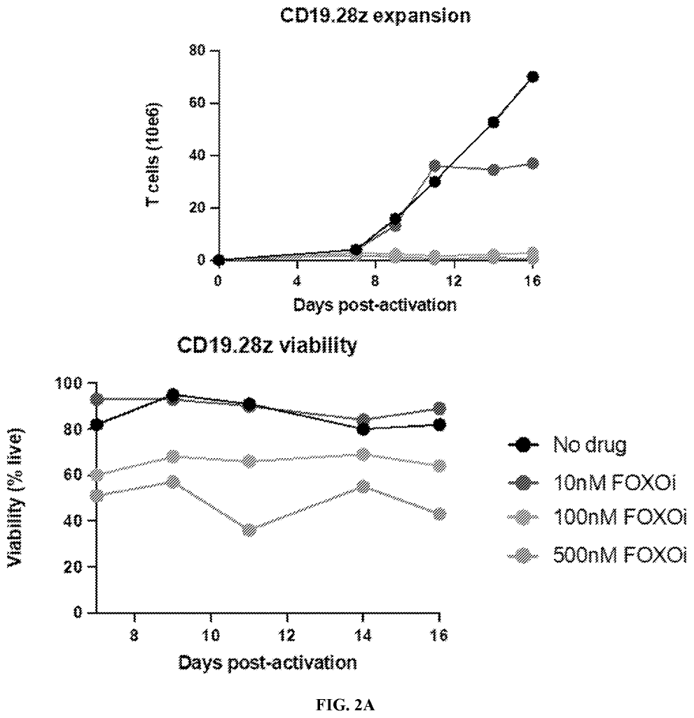

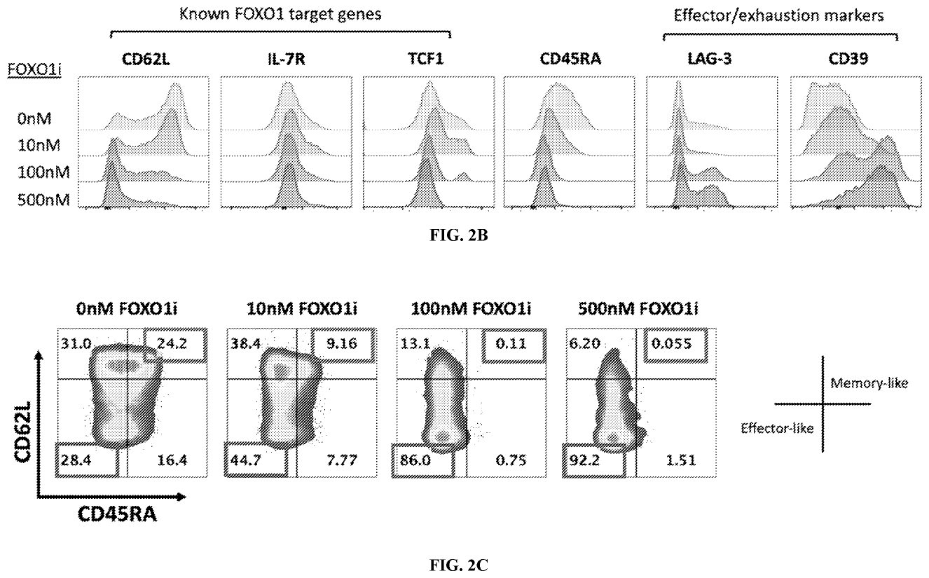

To interrogate FOXO1 function in human CAR-T cells, CAR-T cells were expanded in the presence of a selective FOXO1 small molecule inhibitor (FOXO1i). FOXO1i dose-dependently inhibited CAR-T cell expansion, persistence, and viability (FIG. 2A). Further, FOXO1i dramatically and dose-dependently decreased expression of memory-associated FOXO1 target genes (SEL, IL-7R, TCF7) while concomitantly promoting an effector-like phenotype (FIGS. 2B-2C), indicating that endogenous FOXO1 promotes and maintains a memory phenotype in human CAR-T cells. These studies showed that FOXO1 biology enhanced the persistence and overall potency of engineered T cell therapies.

### Example 3

### Ectopic Expression of TFs Via Retroviral Co-Transduction

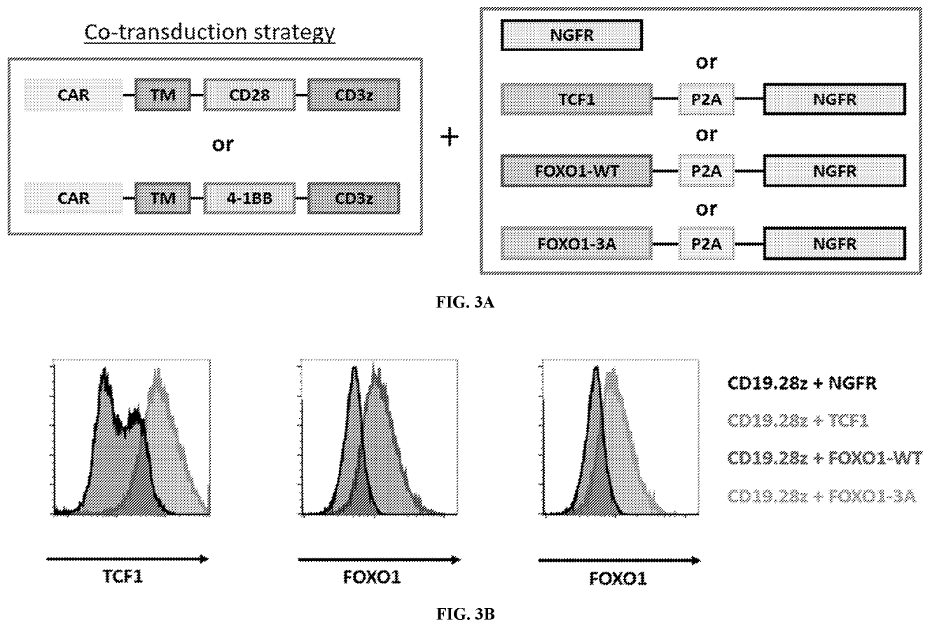

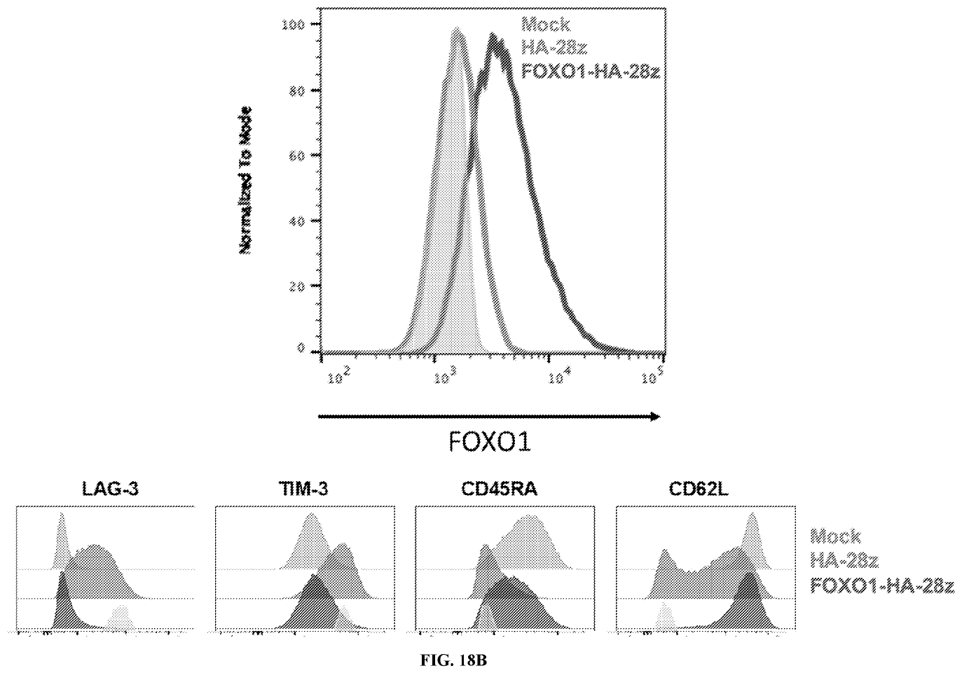

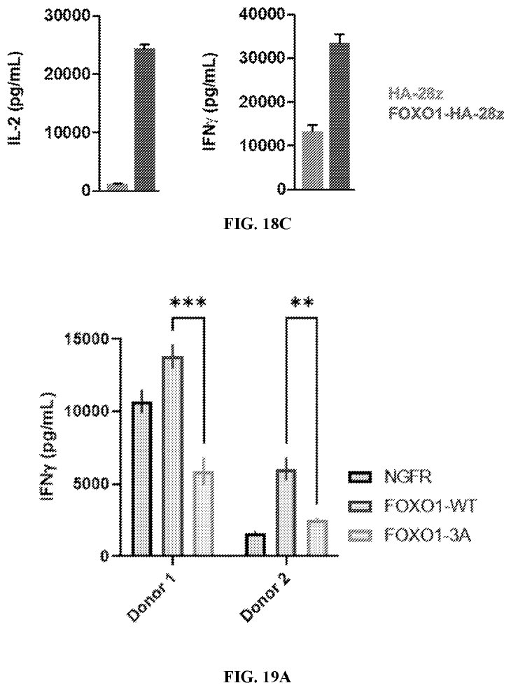

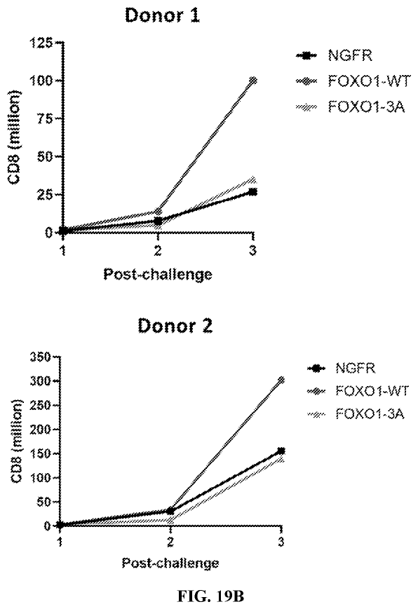

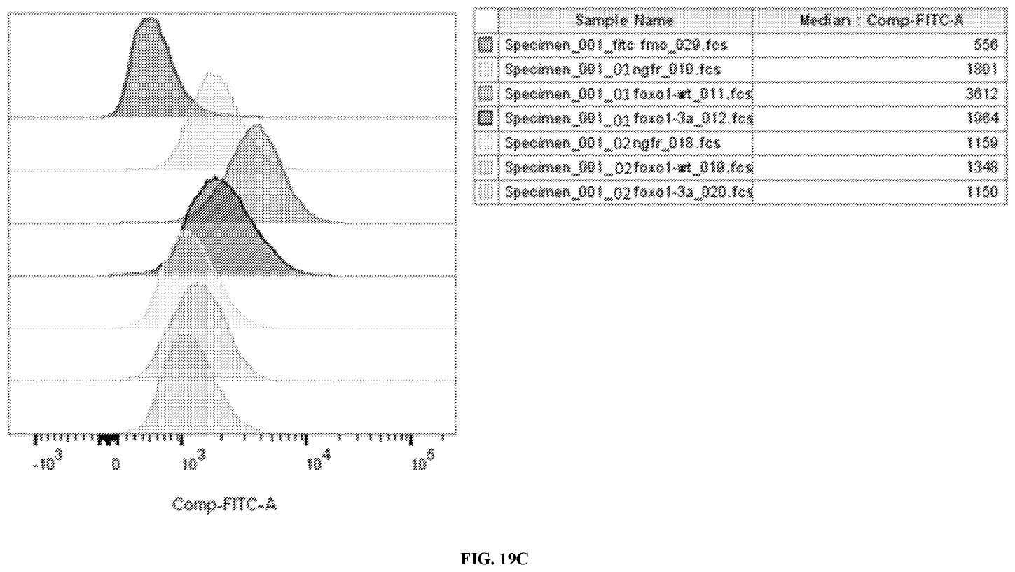

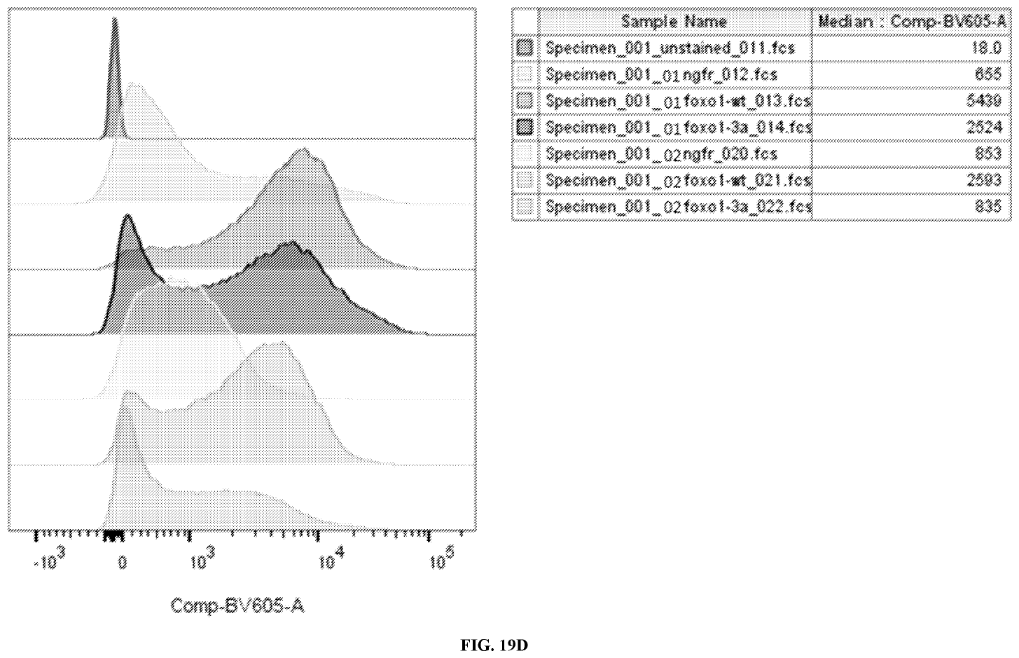

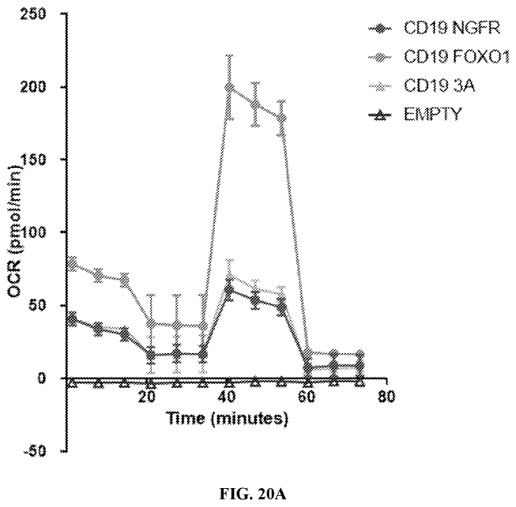

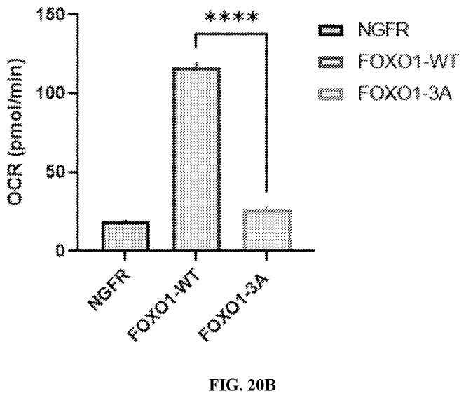

To ectopically express transcription factors (TFs) in primary human T cells, a co-transduction approach was used in which the CAR was expressed using one retrovirus, and a bicistronic vector with the TF plus a truncated nerve growth factor receptor (NGFR) was expressed using a separate retrovirus (FIG. 3A). Control CAR-T cells were co-transduced with a retrovirus solely expressing NGFR. This approach enabled normalization of CAR expression across groups, maximization of TF expression, and identification and/or purification of TF-expressing T cells using NGFR as a surrogate surface marker. 3-5 fold expression was routinely achieved over endogenous levels (FIG. 3B). Additionally, bicistronic vectors expressing both TF and CAR under the same constitutive promoter showed similar results (FIGS. 18A-18C).

### Example 4

### Ectopic Expression of FOXO1-WT and FOXO1-AAA does not Affect CAR-T Cell Function During Short-Term Activation

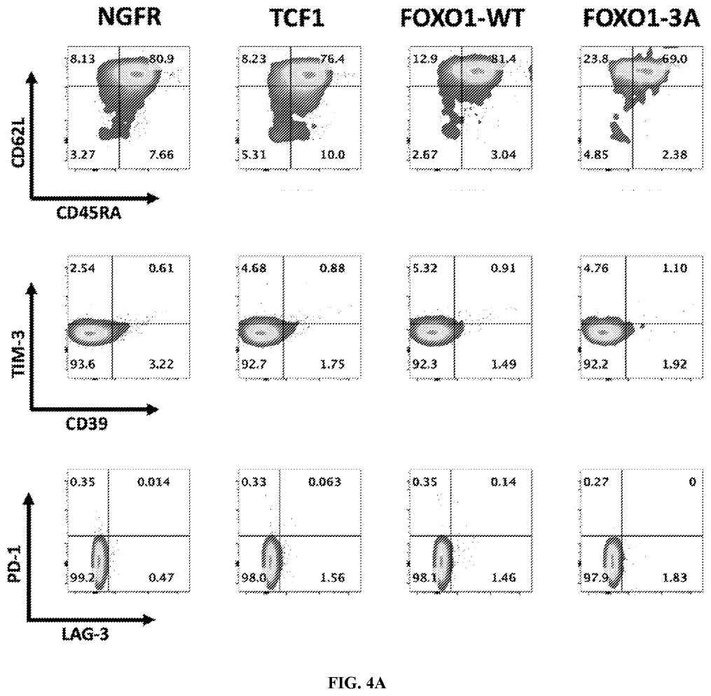

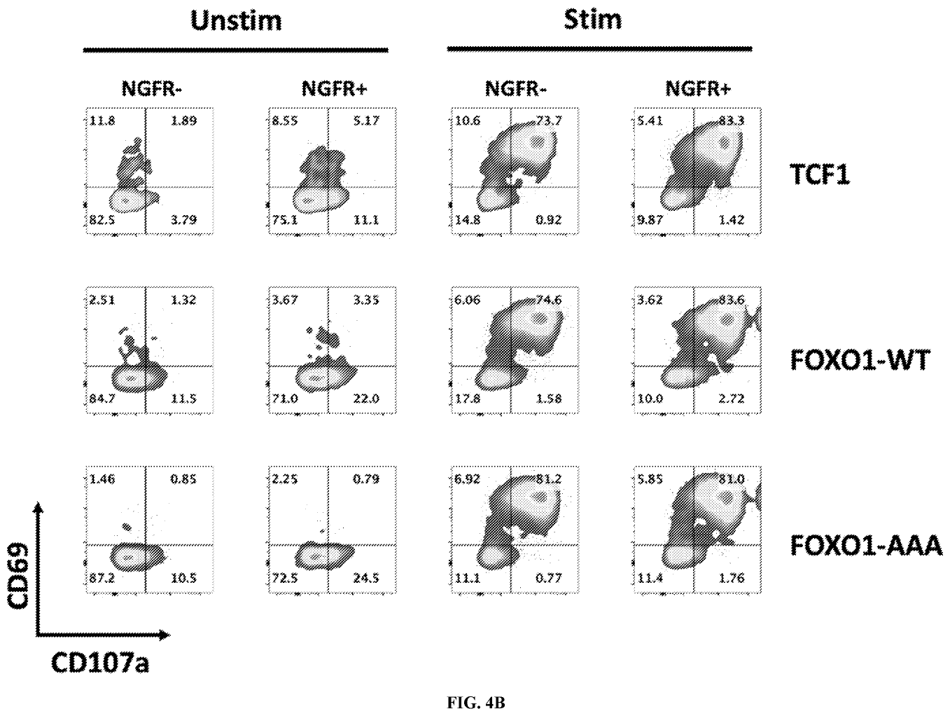

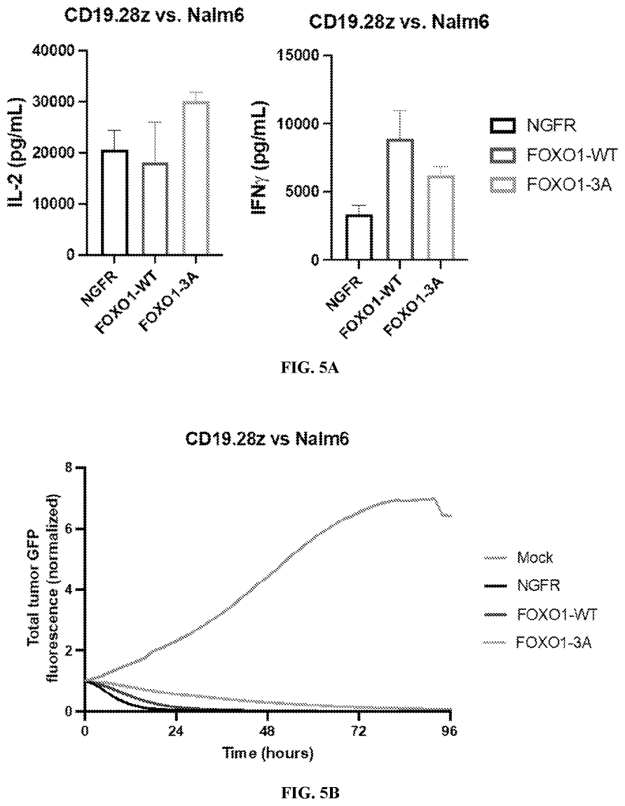

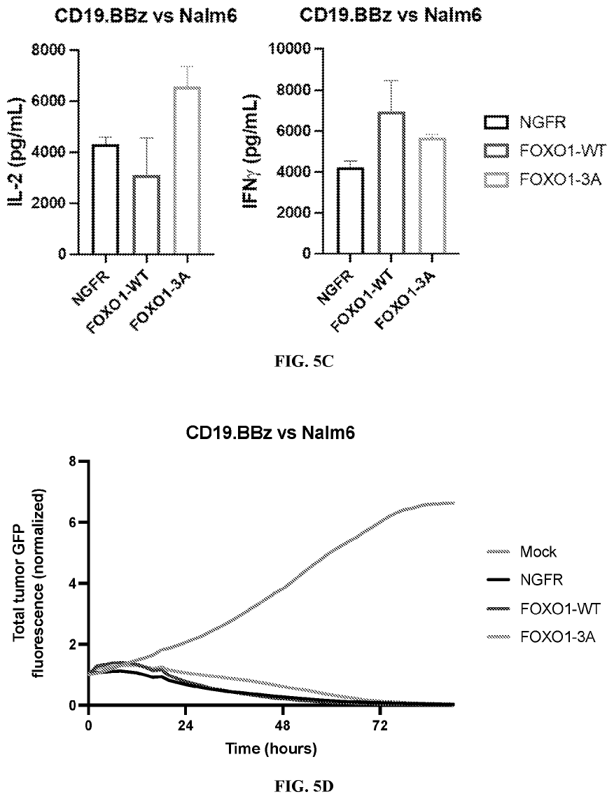

Single cell phenotyping and functional assays were performed on CD19 targeting CAR-T cells with either a CD28 (CD19.28z) or 4-1BB (CD19.BBz) co-stimulatory domains with or without ectopic TF expression. Ectopic expression of FOXO1-WT, FOXO1-3A, and TCF1 did not dramatically affect the pre-stimulation surface phenotype or the extent to which tumor-stimulated CAR-T cells upregulated activation and degranulation markers (CD69 and CD107a, respectively) (FIG. 4), killed, or secreted inflammatory cytokines (FIG. 5). These results showed that ectopic FOXO1 overexpression did not improve nor impede effector function of transiently activated CAR-T cells.

### Example 5

### Ectopic FOXO1 Upregulates Endogenous LEF1 and TCF1 in Resting CAR-T Cells and Sustains their Expression During Activation

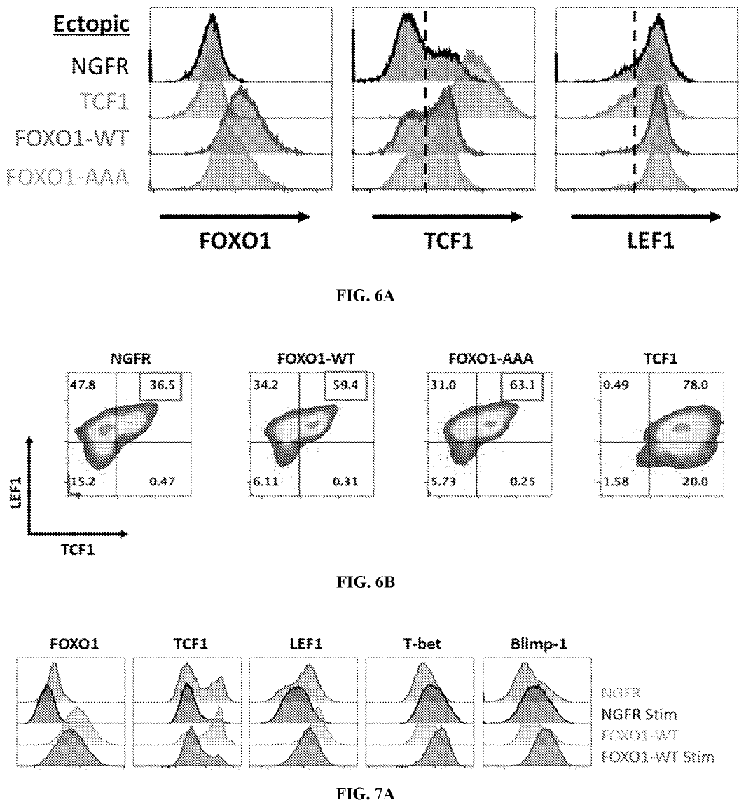

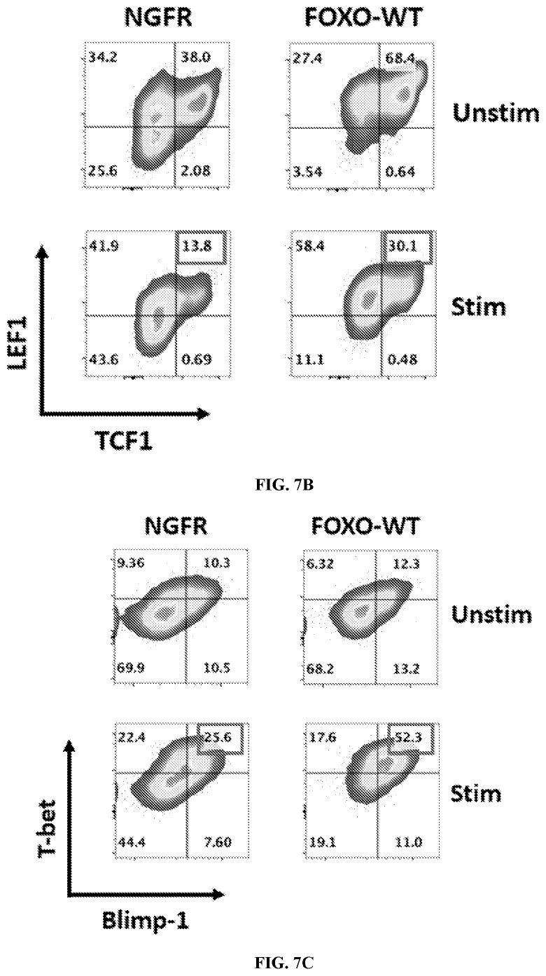

Despite similar surface marker expression of resting FOXO1-overexpressing CD19 CAR-T cells compared to controls (FIG. 4), it was hypothesized that FOXO1-overexpressing CAR-T cells might drive the expression of endogenous TCF1 (gene name TCF7), a memory and stemness-associated TF and FOXO1 target gene that correlates with response to checkpoint blockade and CAR-T cell responses in patients. Indeed, intracellular flow cytometry revealed that ectopic expression of FOXO1-WT and FOXO1-3A enhanced the expression of TCF1 and its related HMG-box family TF, lymphoid enhancer binding factor 1 (LEF1), in resting CD19 CAR-T cells (FIG. 6). Remarkably, the expression of these sternness-associated TFs was sustained during CAR-T cell activation in addition to enhanced expression of effector-associated TFs T box transcription factor TBX21 (Tbet) and B lymphocyte-induced maturation protein-1 (Blimp-1) (FIG. 7). Collectively, these data showed that FOXO1 directly and/or indirectly upregulated TFs that are known to drive stemness and effector transcriptional programs and confer enhanced functionality during settings of chronic stimulation.

### Example 6

### FOXO1-Engineered CD19-Targeting CAR-T Cells Demonstrate Augmented Function in Models of Chronic Antigen Simulation

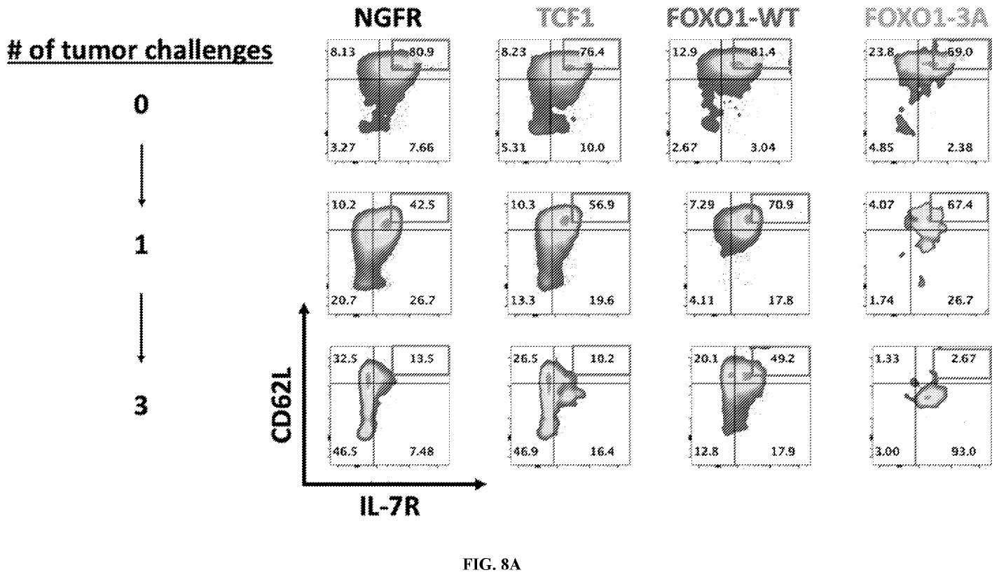

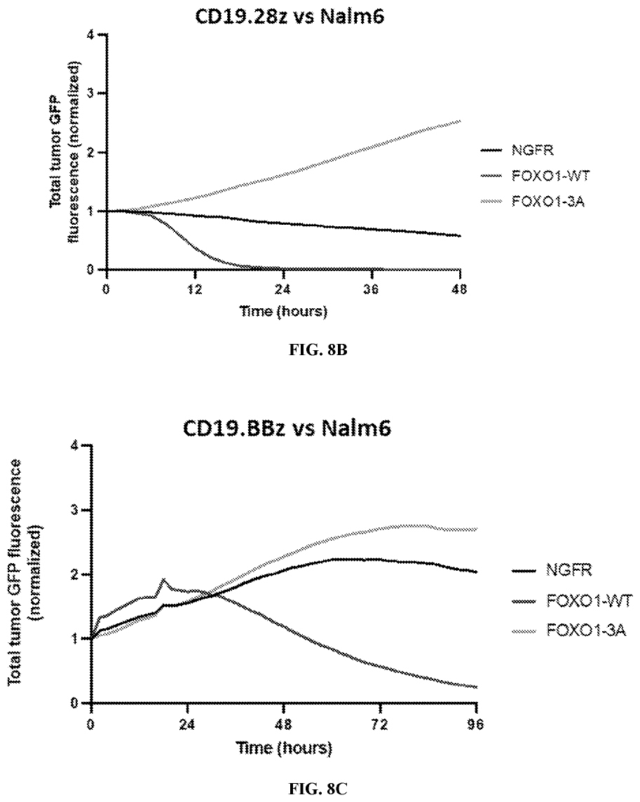

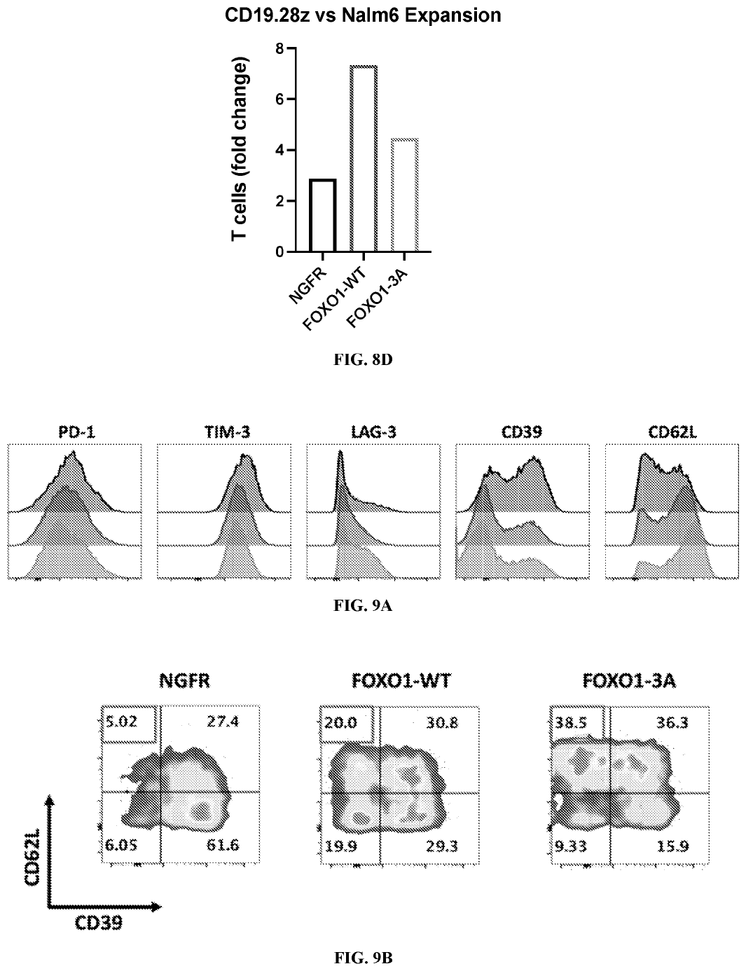

To examine the effects of ectopic FOXO1 expression on CAR-T cell function during chronic stimulation, CD19-targeting CAR-T cells that were co-transduced with either NGFR, FOXO1-WT, or FOXO1-3A were challenged with repeated tumor stimulations or at low effector to tumor (E:T) ratios. After 3 tumor challenges, CD19.BBz CAR-T cells were allowed to rest down for 7 days prior to phenotyping to determine the extent to which FOXO1-engineered CAR-T cells could form memory (which is an important characteristic for sustaining anti-tumor responses). Those expressing FOXO1-WT and FOXO1-3A maintained much higher expression levels of memory markers CD62L and IL-7R compared to TCF1 or NGFR cells, indicating that they had augmented capacity for memory formation and/or persistence (FIG. 8A). For both CD19.28z and CD19.BBz CAR-T cells, ectopic FOXO1-WT, but paradoxically, not FOXO1-3A, enhanced FOXO1 killing compared to NGFR controls (FIGS. 8B-8C). Further interrogation revealed that FOXO1-WT CAR-T cells exhibited enhanced CAR-T cell expansion at low E:T ratios compared to NGFR and FOXO1-3A (FIG. 8D). These data indicated that FOXO1-WT cells were more potent than FOXO1-3A and NGFR cells in settings of chronic antigen stimulation, and that fixed nuclear-localization of FOXO1-3A is constraining CAR-T cell expansion or effector function during chronic stimulation.

### Example 7

### Ectopic FOXO1 Mitigates T Cell Exhaustion

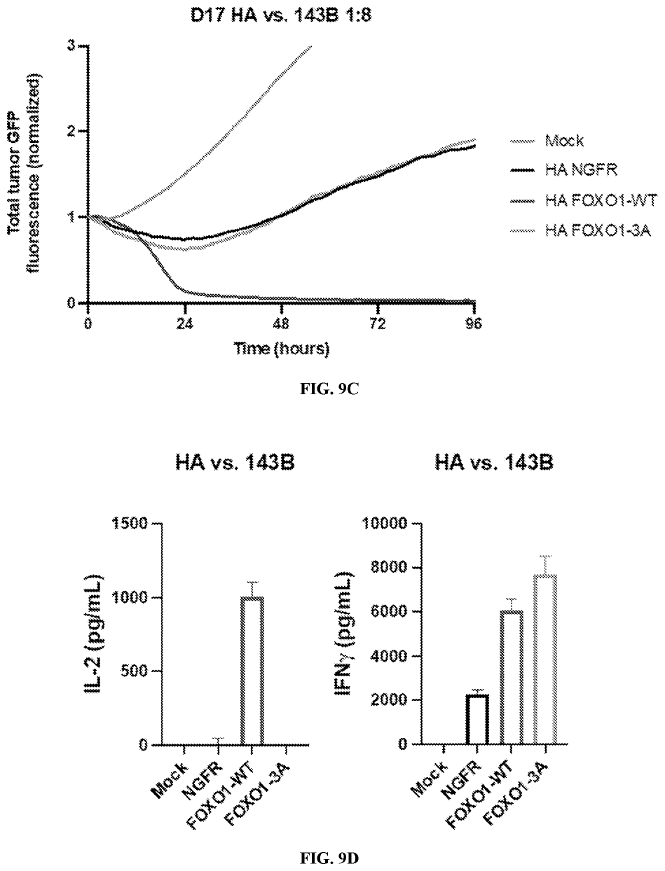

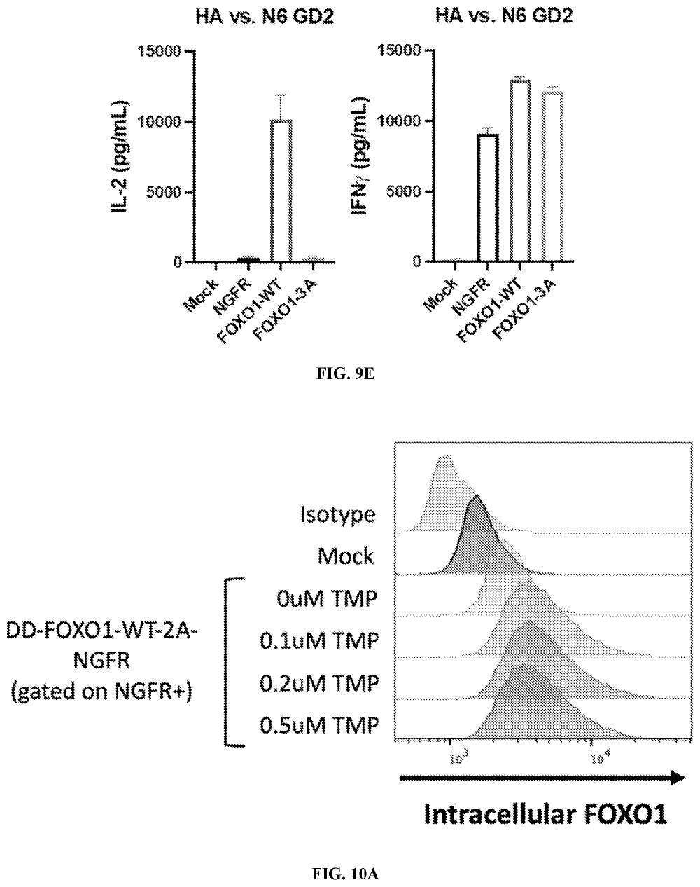

Ectopic TF expression for enhancement of CAR-T cell function was also tested in a validated in vitro model of CAR-T cell exhaustion in which a high-affinity GD2-targeting CAR (HA.28z) promotes antigen-independent aggregation and signaling, and rapid onset of T cell dysfunction. HA.28z CAR-T cells expressing FOXO1-WT and FOXO1-3A both demonstrated an altered surface phenotype in which exhaustion markers (ex. CD39 and PD-1) were reduced, while memory markers and FOXO1 target genes (ex. CD62L) were increased (FIGS. 9A-9B). FOXO1-WT cells demonstrated enhanced cytokine secretion and killing compared to controls; however, FOXO1-3A cells were not functionally enhanced despite exhibiting a surface phenotype consistent with non-exhausted cells (FIGS. 9C-9E), which was consistent with observations from CD19-targeting CARs undergoing chronic stimulation (FIG. 8). Collectively, these data showed that ectopic FOXO1-WT counteracted CAR-T cell exhaustion and endowed enhanced function.

### Example 8

### Constitutive Expression of FOXO1 Confers Exhaustion Resistance

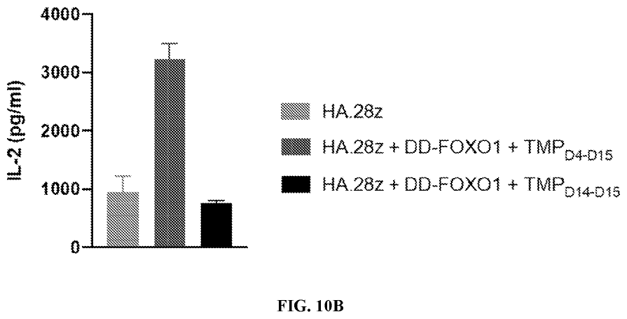

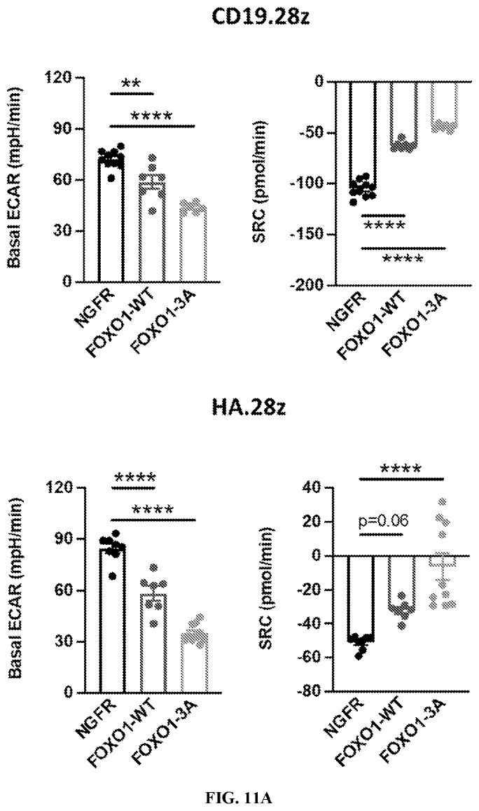

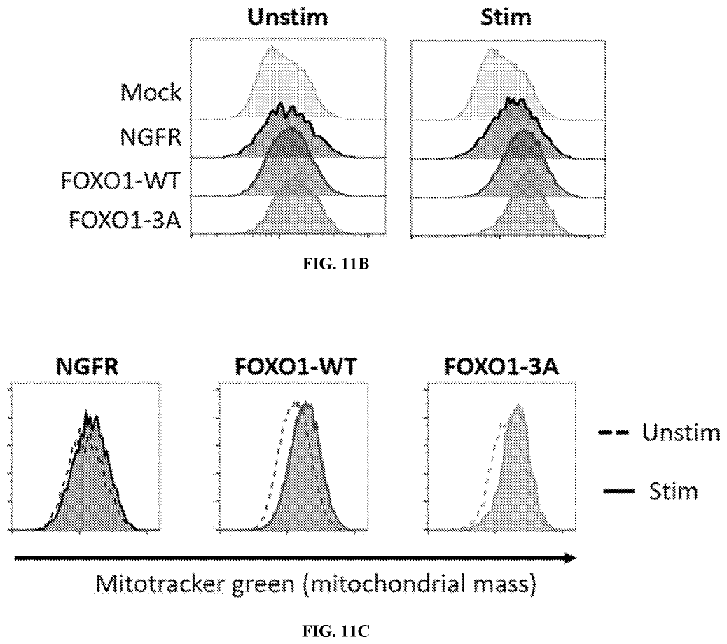

To test whether constitutive FOXO1-WT mitigated exhaustion in the HA.28z CAR model, or whether transient expression at the time of tumor exposure was sufficient to augment function, a destabilization domain was fused to the N terminus of FOXO1-WT (DD-FOXO1-WT), which enabled precise, drug-dependent control of expression (FIG. 10A). HA.28z CAR-T cells expressing DD-FOXO1-WT from day 4 through day 15 demonstrated increased IL-2 secretion in response to tumor, whereas those expressing DD-FOXO1-WT starting 24 hours prior tumor challenge did not (FIG. 10B).

### Example 9

### Ectopic FOXO1 Metabolically Reprograms CAR-T Cells

Since FOXO1 promotes memory and stemness in CAR-T cells, and these phenotypes are known to correlate with differential metabolic features compared to effector T cells, it was hypothesized that ectopic FOXO1 might be driving metabolic reprogramming in CAR-T cells. Using a Seahorse assay, we discovered that both FOXO1-WT and FOXO1-3A lowered the extracellular acidification rate (basal ECAR) and increased mitochondrial fitness (e.g., spare respiratory capacity, SRC) both in resting CD19-targeting and exhausted HA.28z CAR-T cells (FIG. 11A). FOXO1-WT and FOXO1-3A CAR-T cells also exhibited higher mitochondrial mass in the unstimulated (unstim) state than NGFR controls (FIG. 11B). Antigen stimulation further increased mitochondrial mass in FOXO1-engineered CAR-T cells, while NGFR control cells remained at baseline levels (FIG. 11C). Collectively, these data demonstrated that ectopic FOXO1-WT and FOXO1-3A metabolically reprograms CAR-T cells with features that are characteristics of memory T cells.

### Example 10

### FOXO1-Engineered CAR-T Cells Mediate Enhanced Tumor Control and Mouse Survival in Liquid Tumor Models

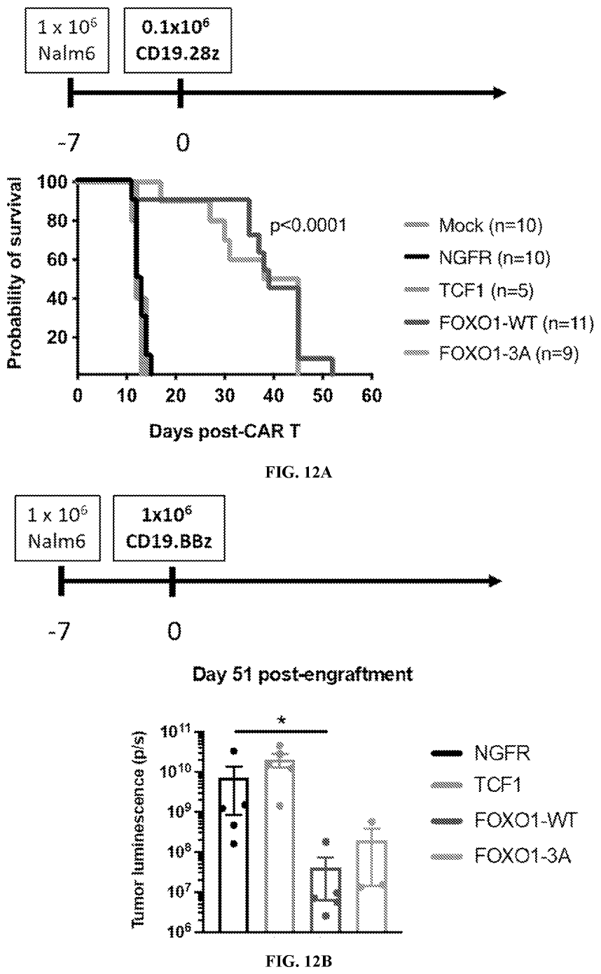

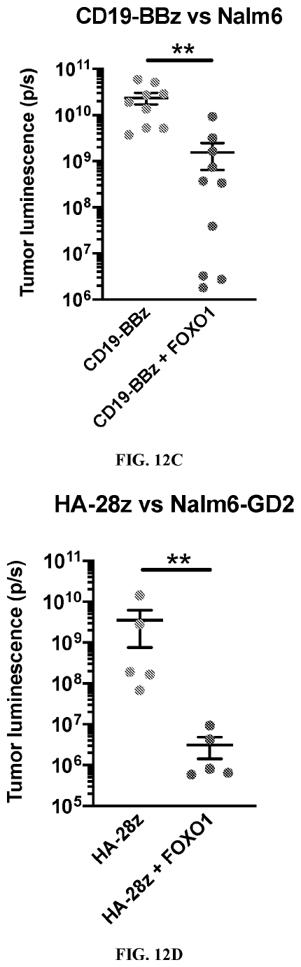

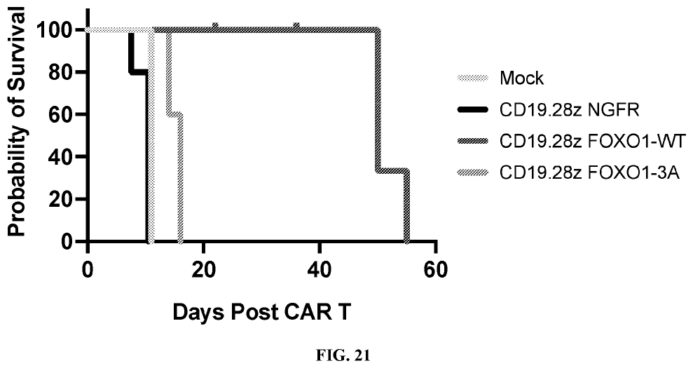

A non-curative dose of TF-engineered or control CAR-T cells (CD19.28z, CD19.BBz, or HA.28z) were infused into mice engrafted with Nalm6 leukemia. FOXO1-WT and CAR-T cells demonstrated markedly enhanced tumor control and survival compared to NGFR control and TCF1 cells (FIGS. 12A-12C, 21). FOXO1-3A CAR-T cells also demonstrated enhanced tumor control and survival compared to NGFR control and TCF1 cells but to a lesser degree than FOXO1-WT and CAR-T cells. These data further supported the notion that ectopic FOXO1 augments CAR-T cell function particularly in settings of chronic antigen exposure by mitigating T cell exhaustion and enforcing stemness programs. Finally, FOXO1-WT HA.28z CAR-T cells, which were functionally superior to NGFR control cells in vitro, demonstrated augmented tumor control in a leukemia xenograft model in which Nalm6 leukemia cells were engineered to express GD2 (FIG. 12D).

### Example 11

### CAR-T Cells Expressing FOXO1-WT Outperform FOXO1-3A in an In Vivo Model of Leukemia Via Enhanced Expansion, Persistence, and Recall Capacity

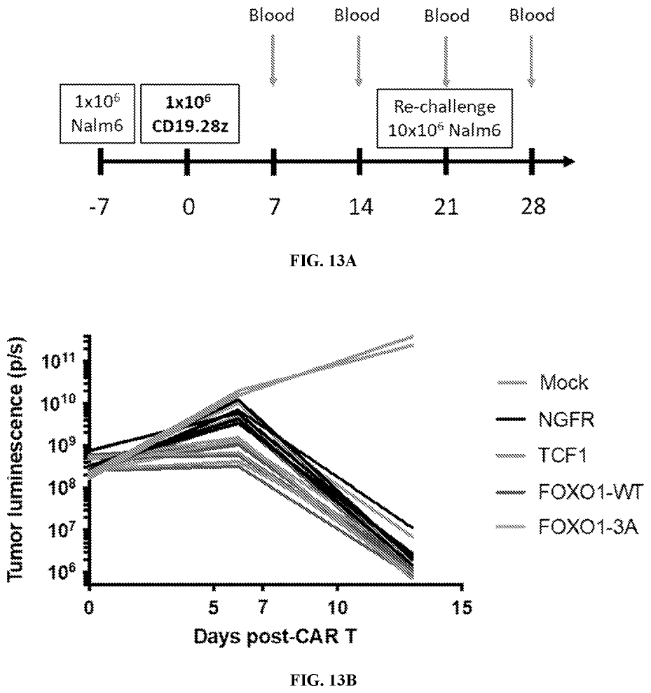

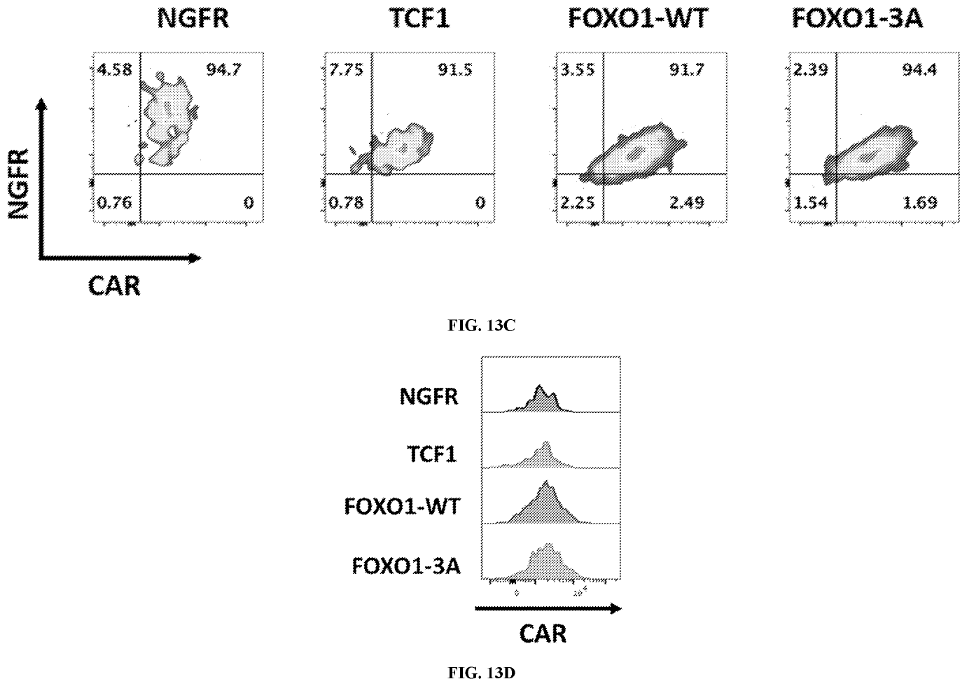

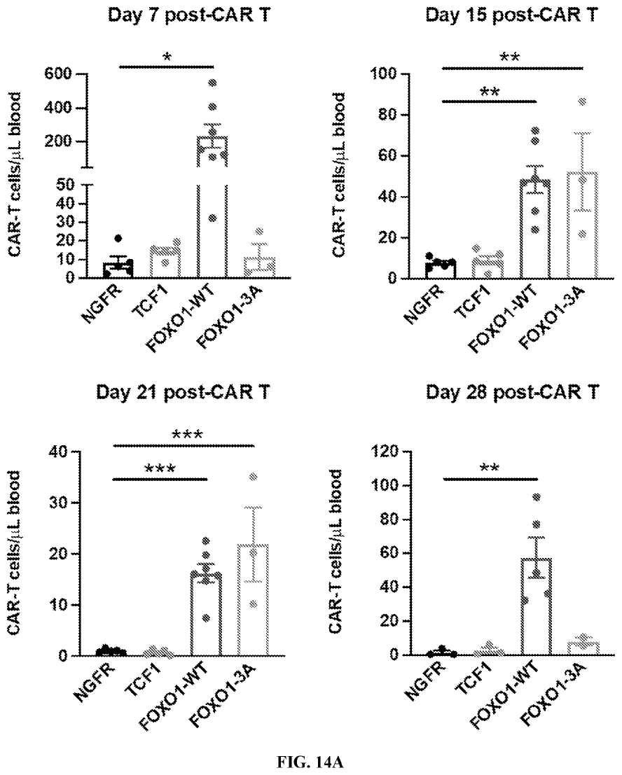

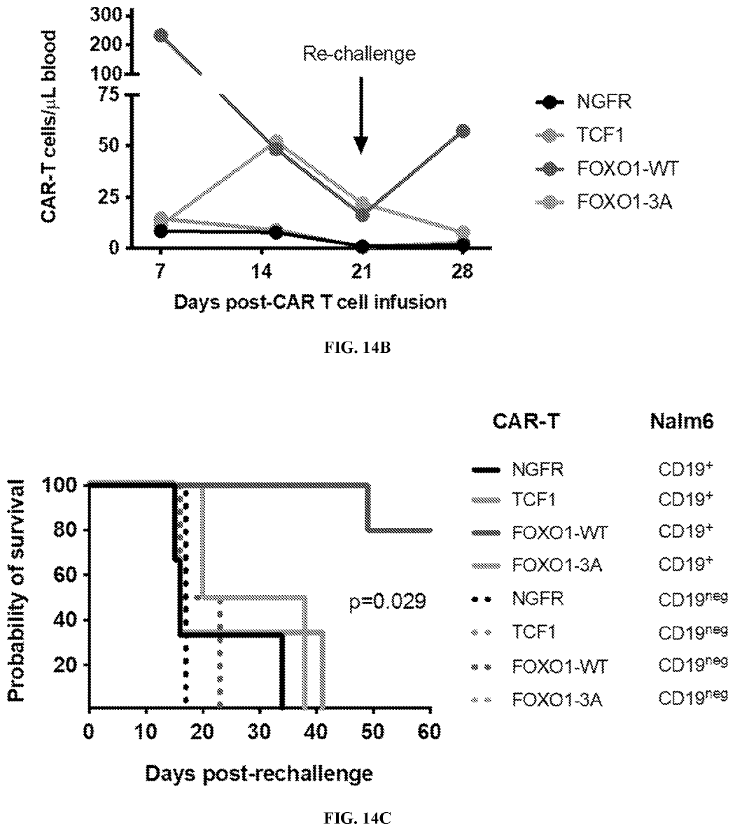

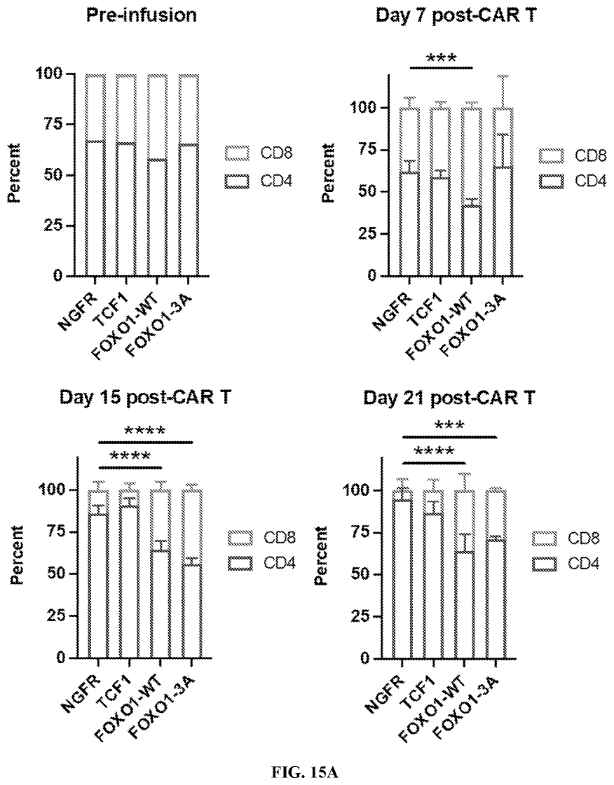

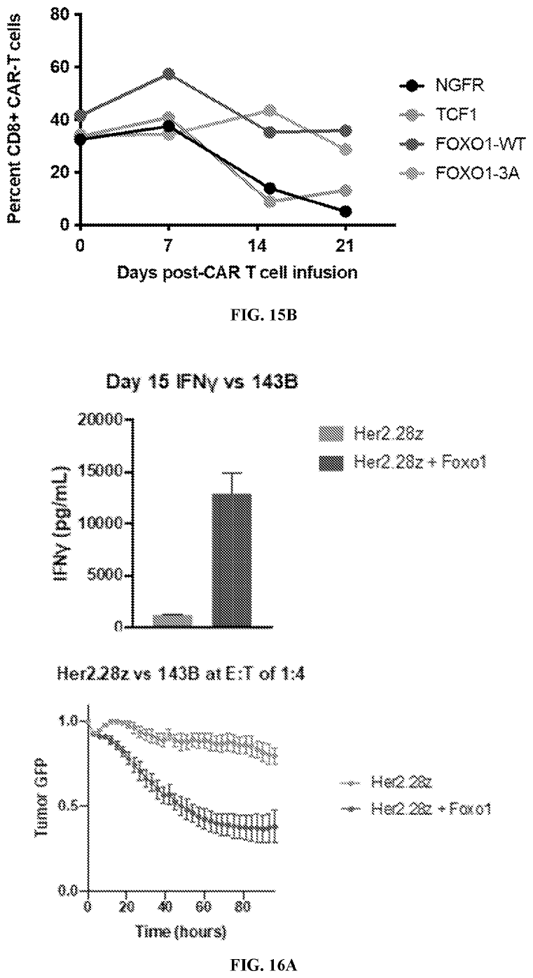

A curative dose of NGFR-purified CD19.28z CAR T cells that co-express ectopic FOXO1-WT, FOXO1-3A, TCF 1, or control NGFR were infused into NSG mice engrafted with Nalm6 leukemia. Circulating levels of CAR-T cells were measured 7 days post-CAR-T infusion and every 7 days thereafter (FIG. 13A). One week after tumors were cleared in all groups (FIG. 13B), mice were rechallenged with a large dose of 10e6 Nalm6 to assess whether persisting CAR-T cells were responsive to a secondary challenge. Circulating CAR-T cells that were detected at each timepoint remained CAR+/NGFR+ (FIG. 13C), and CAR surface density was consistent across experimental groups (FIG. 13D). Remarkably, at day 7 post-infusion, FOXO1-WT cells expanded approximately 28-fold more than NGFR controls and 20-fold more than FOXO1-3A cells (FIGS. 14A-14B). FOXO1-3A cells exhibited robust but delayed expansion, as circulating levels peaked at day 14 post-infusion and far exceeded NGFR control levels but were still 4-5 fold lower than FOXO1-WT levels observed on day 7 (FIGS. 14A-14B). FOXO1-WT and FOXO1-3A CAR-T cells demonstrated equivalent persistence in mice as far out as 21 days, a time point in which control NGFR and TCF1 cells were nearly undetectable (FIGS. 14A-14B). At 7 days post-tumor rechallenge (day 28), only FOXO1-WT cells demonstrated recall capacity, which was characterized by 2-3 fold expansion of circulating CAR-T cells in response to rechallenge and prolonged mouse survival (FIG. 14). In contrast, circulating FOXO1-3A CAR-T cell levels continued to decline after rechallenge and eventually these mice and those treated with control NGFR and TCF1 cells succumbed to disease (FIG. 14). Importantly, at all timepoints, the relative frequency of CD8 CAR-T cells was significantly higher in both FOXO1-WT and FOXO1-3A conditions compared to controls, thereby suggesting a role for FOXO1 in augmenting CD8+ CAR-T cell expansion and persistence (FIG. 15). In summary, FOXO1-WT and FOXO1-3A CAR-T cells demonstrated enhanced expansion and persistence compared to control NGFR and TCF1 cells. However, FOXO1-WT cells expanded more robustly and retained their anti-tumor functionality compared to FOXO1-3A cells, further supporting the notion that ectopic FOXO1-WT enhanced CAR-T cell function to a greater extent than ectopic FOXO1-3A.

### Example 12

### FOXO1-Engineered CAR-T Cells Enhanced CAR-T Cell Targeting to Solid Tumors

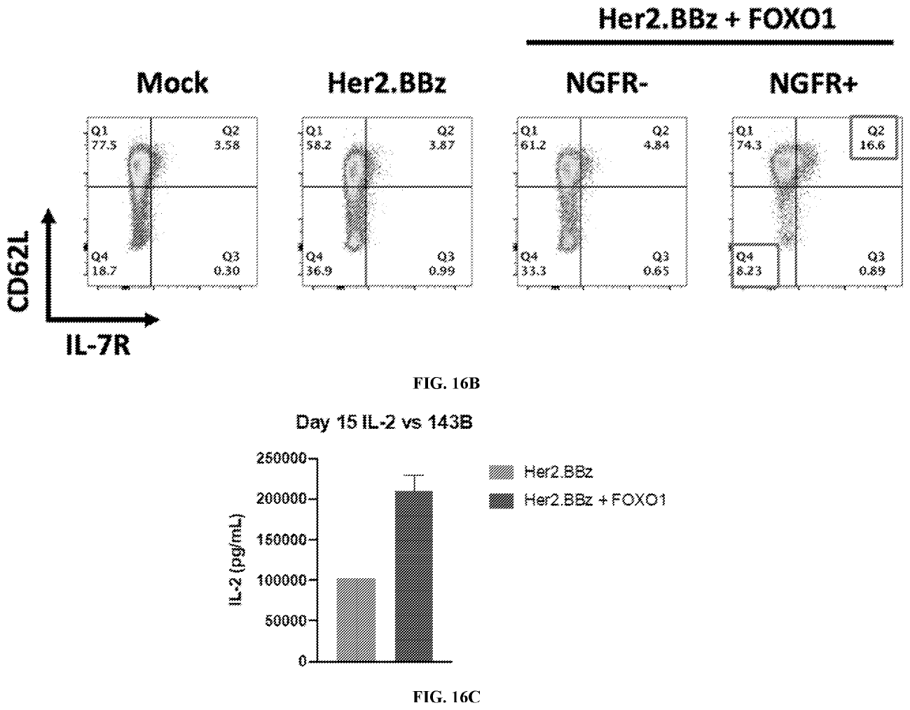

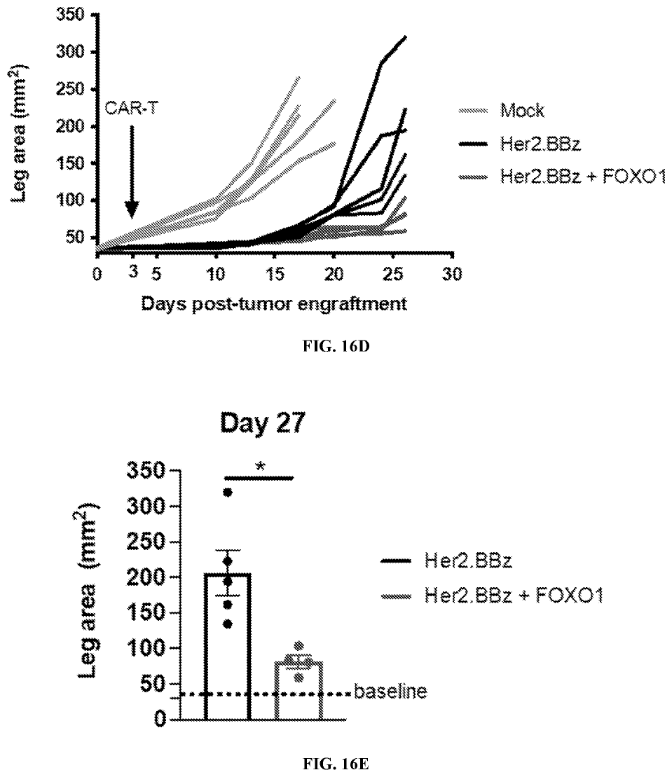

In contrast to CD19-targeting CAR-T cells, where FOXO1-engineered cells phenotypically and functionally resembled control CAR-T cells at rest and during transient activation, ectopic FOXO1-WT in those targeting Her2 (Her2.28z and Her2.BBz) exhibited a higher frequency of memory-like T cells at rest and enhanced function in short-term assays (FIGS. 16A-16C). A non-purified dose of 10e6 CAR-T cells (of which only 50% were NGFR/FOXO1-WT+) were infused into mice engrafted with the osteosarcoma cell line 143B. FOXO1-WT cells augmented tumor control in this extremely aggressive solid tumor model (FIGS. 16D-16E), showing that this approach is widely applicable to solid tumors in addition to hematologic malignancies.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising isolated T cells that comprise an exogenous nucleic acid encoding a forkhead box protein O1 (FOXO1).

2. (canceled)

3. The composition of claim 1, wherein the FOXO1 is fused to a motif which modulates expression levels or enhances intracellular degradation.

4. The composition of claim 1, wherein the isolated T cells maintain functionality under conditions in which unmodified T cells display exhaustion.

5. The composition of claim 1, wherein the isolated T cells further comprise a nucleic acid encoding a recombinant receptor.

6. The composition of claim 5, wherein the recombinant receptor is a T cell receptor (TCR) or a chimeric antigen receptor (CAR).

7. (canceled)

8. The composition of claim 5, wherein the FOXO1 and the engineered receptor are encoded by separate nucleic acids or a single nucleic acid and/or are expressed under different promoters.

9-10. (canceled)

11. The composition of claim 1, wherein the isolated T cells are from a biological sample from a subject.

12. (canceled)

13. The composition of claim 1, wherein the isolated T cells are expanded ex vivo.

14. The composition of claim 1, further comprising at least one therapeutic agent.

15. A method of treating a disease or disorder in a subject comprising administering to the subject having the disease or disorder an effective amount of the composition of claim 1.

16. The method of claim 15, wherein the isolated T cells are autologous to the subject.

17. The method of claim 1, wherein FOXO1 is overexpressed in the isolated T cells prior to exposure to the antigen.

18. The method of claim 15, wherein the disease or disorder comprises an infectious disease or cancer.

19. (canceled)

20. The method of claim 18, wherein the isolated T cells further comprise a nucleic acid encoding a recombinant receptor specific for the cancer.

21. The method of claim 18, wherein the administering reduces the number of cancerous cells in the patient, reduces and/or eliminates the tumor burden in the patient, or a combination thereof and/or shows enhanced cancer treatment compared to administration of unmodified T cells.

22-23. (canceled)

24. The method of claim 18, further comprising administering to the patient one or more chemotherapeutic agents.

25. The method of claim 15, wherein the administering results in lower expression of inhibitory receptors in the subject in comparison to administration of unmodified T cells.

26. A method for preventing exhaustion of engineered T cells comprising introducing a nucleic acid that overexpresses FOXO1 into the engineered T cells.

27-28. (canceled)

29. The method of claim 26, wherein the engineered T cells further comprise a nucleic acid encoding a recombinant receptor, wherein the FOXO1 and the engineered receptor are encoded by separate nucleic acids or a single nucleic acid and/or are expressed under different promoters.

30-35. (canceled)

36. The method of claim 26, further comprising administering the T cells to a subject in need thereof.

37-39. (canceled)
